SEQUENCE LISTING (40 sequences)

SEQ ID NO: 1 - DNA, 169 bp, unidentified
  source 1..169: mol_type = other DNA
tgcgcacccttagcgagaggtttatcattaaggtcaacctctggatgttgtttcggcatcctgcattgaatctgagttactgtctgttttccttgttggaacggagagcatcgcctgatgctctccgagccaaccaggaaacccgttttttctgacgtaagggtgcgca

SEQ ID NO: 2 - DNA, 963 bp, synthetic construct
  source 1..963: mol_type = other DNA
atgaaatctgcagagtatctgaatacgttccgccttaggaatttgggcctccccgtgatgaacaatctccacgatatgagcaaggcgactcgaatatccgtggaaacgctgagactgctcatctatacagcagactttcggtacaggatctacacggtcgaaaagaaggggcctgagaaacgcatgcgaacaatttatcaacctagccgagagctcaaggcgttgcagggctgggttcttcgaaacatccttgacaaactctcatcatcaccctttagtattgggtttgaaaagcaccaaagcatccttaacaacgcgacgccacacataggtgccaatttcatattgaacatcgacttggaggatttttttccgagcctcacagccaataaagtgttcggtgtttttcacagtcttgggtacaatcgccttattagttccgttcttaccaagatttgttgttacaagaatctcttgccccagggagcacccagcagtccgaaattggcgaatttgatttgttccaagctcgattatcgaatacaagggtacgcgggcagccggggactcatctatacccgctacgcagacgatcttacgctgtctgcccaatcaatgaagaaggtcgtaaaggcgcgggatttcttgttttctatcatcccgtccgagggcttggtaattaattccaaaaagacttgtatctcaggaccacgatctcagcgaaaagtgacaggactcgtcatttctcaagaaaaagtcggtatagggagagagaagtataaggaaatccgcgcgaagatccaccacatattctgtggcaagagcagcgagatagaacacgtccgaggctggttgtccttcatactgagcgtggactcaaaaagccaccgccggttgatcacctatatttcaaaactggaaaagaaatatggaaagaacccactcaacaaagctaaaacatag

SEQ ID NO: 3 - DNA, 1761 bp, synthetic construct
  source 1..1761: mol_type = other DNA
atgacaaaaacttcaaagctggatgcgctgcgggcggctactagtagggaagatttggcgaagattctcgacataaagttggtgtttctgacaaacgtgttgtaccgcataggatccgacaaccagtatacgcaattcacaatacccaaaaagggtaaaggtgtccgcaccatcagcgcaccaacggaccgacttaaggatatacagaggaggatttgtgatcttcttagtgactgtagggatgaaatctttgcgattaggaagatctctaataattactcattcggcttcgaaagaggaaaatcaattatactcaatgcttacaagcatcgagggaagcaaattatattgaacatcgaccttaaggacttctttgagagctttaactttgggagagtccgggggtactttctctccaaccaggacttcttgttgaacccagttgtggcaacaacgttggcgaaggccgcctgctacaacgggactctgcctcaggggtccccatgttcccctattataagtaaccttatctgtaacattatggacatgcggctcgcaaagctcgccaagaagtacggctgcacttatagtcgatatgcggatgacattacgatcagcaccaataaaaataccttcccgttggagatggcgactgtgcagcctgaaggggttgtgctgggcaaagtgctcgtaaaggagattgaaaattcaggtttcgagattaacgattctaagactagattgacctacaaaacaagtaggcaagaagtcaccgggctgacggttaatcggattgtaaacattgatcggtgctactacaaaaagacgagggcgctggctcacgcattgtatcggacaggagaatataaggtcccagacgagaacggtgttctggtatctggagggcttgacaagttggagggtatgtttgggtttatcgaccaggtggataaattcaacaacattaaaaaaaagttgaataagcaacccgacagatatgttctgacaaatgccactttgcacggatttaagctcaaattgaacgccagggagaaagcctatagcaaattcatctactacaaattcttccacggtaatacttgtcccacgatcataacagagggtaagacggataggatttaccttaaagctgccctccatagcctcgagacaagttatcctgaactgtttcgggagaaaacagatagtaagaagaaggagataaatctgaatatttttaaaagcaatgagaagaccaagtatttcctggatctcagcggcggcacagcagacctcaagaaattcgtggaacgctacaaaaataactacgcttcctattacggcagcgtaccgaaacaaccggtgataatggtgcttgataacgacacaggcccgtcagacctgttgaactttttgagaaacaaagttaagagttgtccagatgatgtaacagaaatgcgcaagatgaagtacatacatgtgttttacaatctgtacatagttctgactcccctgtctccatctggagagcaaacgtctatggaggacctctttcctaaagatatattggacattaagatagatggcaagaaattcaataaaaacaatgacggtgactccaaaacagagtatgggaagcacatattctcaatgcgcgttgtacgagataaaaagaggaagatagatttcaaggcattttgctgtatcttcgatgctattaaggatattaaagaacattacaaactgatgttgaattcctag

SEQ ID NO: 4 - protein, 319 aa, unidentified
KSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKT

SEQ ID NO: 5 - protein, 586 aa, Syacium guineensis
MTKTSKLDALRAATSREDLAKILDIKLVFLTNVLYRIGSDNQYTQFTIPKKGKGVRTISAPTDRLKDIQRRICDLLSDCRDEIFAIRKISNNYSFGFERGKSIILNAYKHRGKQIILNIDLKDFFESFNFGRVRGYFLSNQDFLLNPVVATTLAKAACYNGTLPQGSPCSPIISNLICNIMDMRLAKLAKKYGCTYSRYADDITISTNKNTFPLEMATVQPEGVVLGKVLVKEIENSGFEINDSKTRLTYKTSRQEVTGLTVNRIVNIDRCYYKKTRALAHALYRTGEYKVPDENGVLVSGGLDKLEGMFGFIDQVDKFNNIKKKLNKQPDRYVLTNATLHGFKLKLNAREKAYSKFIYYKFFHGNTCPTIITEGKTDRIYLKAALHSLETSYPELFREKTDSKKKEINLNIFKSNEKTKYFLDLSGGTADLKKFVERYKNNYASYYGSVPKQPVIMVLDNDTGPSDLLNFLRNKVKSCPDDVTEMRKMKYIHVFYNLYIVLTPLSPSGEQTSMEDLFPKDILDIKIDGKKFNKNNDGDSKTEYGKHIFSMRVVRDKKRKIDFKAFCCIFDAIKDIKEHYKLMLNS

SEQ ID NO: 6 - protein, 312 aa, synthetic construct
MSIDIETTLQKAYPDFDVLLKSRPATHYKVYKIPKRTIGYRIIAQPTPRVKAIQRDIIEILKQHTHIHDAATAYVDGKNILDNAKIHQSSVYLLKLDLVNFFNKITPELLFKALARQKVDISDTNKNLLKQFCFWNRTKRKNGALVLSVGAPSSPFISNIVMSSFDEEISSFCKENKISYSRYADDLTFSTNERDVLGLAHQKVKTTLIRFFGTRIIINNNKIVYSSKAHNRHVTGVTLTNNNKLSLGRERKRYITSLVFKFKEGKLSNVDINHLRGLIGFAYNIEPAFIERLEKKYGESTIKSIKKYSEGG

SEQ ID NO: 7 - protein, 311 aa, unidentified
MDILQHISDLLLTKKSEIISFSLTAPYRYKIYKIAKRNSDKKRTIAHPSKELKFIQREITEYLTDKLPVHECAFAYKKGSSIKTNAQVHLHTKYLLKMDFENFFPSITPRLFFSKLRLANIDLTADDKVLLENILFFKSKRNSNLRLSIGAPSSPLISNFVMYFWDIEVQEICSKIGVNYTRYADDLTFSTNNKDVLFDIPDMLENVLPKYSLGRIRINHEKTVFSSKGHNRHVTGITLTNDNKLSIGRERKRKISAMIHHFINGKLSTDECNKLVGLLAFAKNIEPSFYKSMVIKYGSDNIYKLQKQKDK

SEQ ID NO: 8 - DNA, 169 bp, synthetic construct; identical to SEQ ID NO: 1
  source 1..169: mol_type = other DNA

SEQ ID NO: 9 - DNA, 199 bp, synthetic construct
  source 1..199: mol_type = other DNA
tgataagattccgtatgcgcacccttagcgagaggtttatcattaaggtcaacctctggatgttgtttcggcatcctgcattgaatctgagttactgtctgttttccttgttggaacggagagcatcgcctgatgctctccgagccaaccaggaaacccgttttttctgacgtaagggtgcgcatacggaatcttatca

SEQ ID NO: 10 - DNA, 228 bp, synthetic construct
  source 1..228: mol_type = other DNA
agctagacttgtttactttgtatttattagtatttgcctcacatgagaacacacactctccatcctgcgtacgccatccccttcgatgcgagtgcgaatcgcggcgtcttggtagcacggcagactggctcactggacgaagagtgcatccgagaaccagaaacaagcaacgttccagtgagttgttccacacatcctttaaagtttagcgtatagaaaattcgagac

SEQ ID NO: 11 - DNA, 18 bp, synthetic construct
  source 1..18: mol_type = other DNA
cgatgcgagtgcgaatcg

SEQ ID NO: 12 - DNA, 18 bp, synthetic construct
  source 1..18: mol_type = other DNA
gtgcgaatcgcggcgtct

SEQ ID NO: 13 - DNA, 18 bp, synthetic construct
  source 1..18: mol_type = other DNA
cgcggcgtcttggtagca

SEQ ID NO: 14 - DNA, 18 bp, synthetic construct
  source 1..18: mol_type = other DNA
cttggtagcacggcagac

SEQ ID NO: 15 - DNA, 18 bp, synthetic construct
  source 1..18: mol_type = other DNA
cacggcagactggctcac

SEQ ID NO: 16 - DNA, 127 bp, synthetic construct
  source 1..127: mol_type = other DNA
tgcgcacccttagcgagaggtttatcattaaggtcaacctctggatgttgtttcggcatcctgcattgaatctgagttactgtctgttttcctaggaaacccgttttttctgacgtaagggtgcgca

SEQ ID NO: 17 - DNA, 127 bp, synthetic construct
  source 1..127: mol_type = other DNA
tgcgcacccttagcgagaggtttatcattaaggtcaacctctggatgttgtttcggcatcctgcattgaatctgagttactgtctgttttcctaggaaacccgtttcttctgacgtaagggtgcgca

SEQ ID NO: 18 - DNA, 157 bp, synthetic construct
  source 1..157: mol_type = other DNA
tgataagattccgtatgcgcacccttagcgagaggtttatcattaaggtcaacctctggatgttgtttcggcatcctgcattgaatctgagttactgtctgttttcctaggaaacccgtttcttctgacgtaagggtgcgcatacggaatcttatca

SEQ ID NO: 19 - DNA, 148 bp, synthetic construct
  source 1..148: mol_type = other DNA
aagattccgtatgcgcacccttagcgagaggtttatcattaaggtcaacctctggatgttgtttcggcatcctgcattgaatctgagttactgtctgttttcctaggaaacccgtttcttctgacgtaagggtgcgcatacggaatct

SEQ ID NO: 20 - DNA, 141 bp, synthetic construct
  source 1..141: mol_type = other DNA
ttccgtatgcgcacccttagcgagaggtttatcattaaggtcaacctctggatgttgtttcggcatcctgcattgaatctgagttactgtctgttttcctaggaaacccgtttcttctgacgtaagggtgcgcatacggaa

SEQ ID NO: 21 - DNA, 135 bp, synthetic construct
  source 1..135: mol_type = other DNA
cgtatgcgcacccttagcgagaggtttatcattaaggtcaacctctggatgttgtttcggcatcctgcattgaatctgagttactgtctgttttcctaggaaacccgtttcttctgacgtaagggtgcgcatacg

SEQ ID NO: 22 - DNA, 156 bp, synthetic construct
  source 1..156: mol_type = other DNA
tgataagattccgtatgcgcacccttagcgagaggtttatcattaaggtcaacctctggatgttgtttcggcatcctgcattgaatctgagttactgtctgttttcctggaaacccgtttcttctgacgtaagggtgcgcatacggaatcttatca

SEQ ID NO: 23 - DNA, 155 bp, synthetic construct
  source 1..155: mol_type = other DNA
tgataagattccgtatgcgcacccttagcgagaggtttatcattaaggtcaacctctggatgttgtttcggcatcctgcattgaatctgagttactgtctgttttcctaggacccgtttcttctgacgtaagggtgcgcatacggaatcttatca

SEQ ID NO: 24 - DNA, 155 bp, synthetic construct
  source 1..155: mol_type = other DNA
tgataagattccgtatgcgcacccttagcgagaggtttatcattaaggtcaacctctggatgttgtttcggcatcctgcattgaatctgagttactgtctgttttcctaggaaccgtttcttctgacgtaagggtgcgcatacggaatcttatca

SEQ ID NO: 25 - DNA, 157 bp, synthetic construct
  source 1..157: mol_type = other DNA
tgataagattccgtatgcgcacccttagcgagaggtttatcattaaggtcaacctctggatgttgtttcggcatcctgcattgaatctgagttactgtctgttttcctaggaaacctgtttcttctgacgtaagggtgcgcatacggaatcttatca

SEQ ID NO: 26 - DNA, 157 bp, synthetic construct
  source 1..157: mol_type = other DNA
tgataagattccgtatgcgcacccttagcgagaggtttatcattaaggtcaacctctggatgttgtttcggcatcctgcattgaatctgagttactgtctgttttcctaggaaacccgtatcttctgacgtaagggtgcgcatacggaatcttatca

SEQ ID NO: 27 - DNA, 157 bp, synthetic construct
  source 1..157: mol_type = other DNA
tgataagattccgtatgcgcacccttagcgagaggtttatcattaaggtcaacctctggatgttgtttcggcatcctgcattgaatctgagttactgtctgttttccttatcatgtaatactctacggcgtaagggtgcgcatacggaatcttatca

SEQ ID NO: 28 - DNA, 157 bp, synthetic construct
  source 1..157: mol_type = other DNA
tgataagattccgtatgcgcacccttagcgagaggtttatcattaaggtcaacctctggatgttgtttcggcatcctgcattgaatctgagttactgtctgttttcctatatcggcgtaatttcggttcgtaagggtgcgcatacggaatcttatca

SEQ ID NO: 29 - DNA, 157 bp, synthetic construct
  source 1..157: mol_type = other DNA
tgataagattccgtatgcgcacccttagcgagaggtttatcattaaggtcaacctctggatgttgtttcggcatcctgcattgaatctgagttactgtctgttttcctatctatcgtgatattcagatcgtaagggtgcgcatacggaatcttatca

SEQ ID NO: 30 - DNA, 157 bp, synthetic construct
  source 1..157: mol_type = other DNA
tgataagattccgtatgcgcacccttagcgagaggtttatcattaaggtcaacctctggatgttgtttcggcatcctgcattgaatctgagttactgtctgttttcctatatggacacggtgagcactcgtaagggtgcgcatacggaatcttatca

SEQ ID NO: 31 - DNA, 157 bp, synthetic construct
  source 1..157: mol_type = other DNA
tgataagattccgtatgcgcacccttagcgagaggtttatcattaaggtcaacctctggatgttgtttcggcatcctgcattgaatctgagttactgtctgttttcctatcatttacggtacgatcagcgtaagggtgcgcatacggaatcttatca

SEQ ID NO: 32 - DNA, 157 bp, synthetic construct
  source 1..157: mol_type = other DNA
tgataagattccgtatgcgcacccttagcgagaggtttatcattaaggtcaacctctggatgttgtttcggcatcctgcattgaatctgagttactgtctgttttcctaggaaatccaacattcactgcgtaagggtgcgcatacggaatcttatca

SEQ ID NO: 33 - DNA, 157 bp, synthetic construct
  source 1..157: mol_type = other DNA
tgataagattccgtatgcgcacccttagcgagaggtttatcattaaggtcaacctctggatgttgtttcggcatcctgcattgaatctgagttactgtctgttttcctggatcactcactgtataccgcgtaagggtgcgcatacggaatcttatca

SEQ ID NO: 34 - DNA, 157 bp, synthetic construct
  source 1..157: mol_type = other DNA
tgataagattccgtatgcgcacccttagcgagaggtttatcattaaggtcaacctctggatgttgtttcggcatcctgcattgaatctgagttactgtctgttttcctaggaaactgtaaataaaccgcgtaagggtgcgcatacggaatcttatca

SEQ ID NO: 35 - DNA, 157 bp, synthetic construct
  source 1..157: mol_type = other DNA
tgataagattccgtatgcgcacccttagcgagaggtttatcattaaggtcaacctctggatgttgtttcggcatcctgcattgaatctgagttactgtctgttttcctgcgggcggtgtaacgttccacgtaagggtgcgcatacggaatcttatca

SEQ ID NO: 36 - DNA, 157 bp, synthetic construct
  source 1..157: mol_type = other DNA
tgataagattccgtatgcgcacccttagcgagaggtttatcattaaggtcaacctctggatgttgtttcggcatcctgcattgaatctgagttactgtctgttttcctaggaaactgtagataacccgcgtaagggtgcgcatacggaatcttatca

SEQ ID NO: 37 - DNA, 161 bp, synthetic construct
  source 1..161: mol_type = other DNA
tgataagattccgtatgcgcacccttagcgagaggtttatcattaaggtcaacctctggatgttgtttcggcatcctgcattgaatctgagttactgtctgttttccttgccaggaaacccgtttcttctgacgtaagggtgcgcatacggaatcttatca

SEQ ID NO: 38 - DNA, 165 bp, synthetic construct
  source 1..165: mol_type = other DNA
tgataagattccgtatgcgcacccttagcgagaggtttatcattaaggtcaacctctggatgttgtttcggcatcctgcattgaatctgagttactgtctgttttccttgttaaccaggaaacccgtttcttctgacgtaagggtgcgcatacggaatcttatca

SEQ ID NO: 39 - DNA, 169 bp, synthetic construct
  source 1..169: mol_type = other DNA
tgataagattccgtatgcgcacccttagcgagaggtttatcattaaggtcaacctctggatgttgtttcggcatcctgcattgaatctgagttactgtctgttttccttgttggccaaccaggaaacccgtttcttctgacgtaagggtgcgcatacggaatcttatca

SEQ ID NO: 40 - DNA, 173 bp, synthetic construct
  source 1..173: mol_type = other DNA
tgataagattccgtatgcgcacccttagcgagaggtttatcattaaggtcaacctctggatgttgtttcggcatcctgcattgaatctgagttactgtctgttttccttgttggaaagccaaccaggaaacccgtttcttctgacgtaagggtgcgcatacggaatcttatca

(19) United States
(12) Patent Application Publication
(10) Pub. No.: US 2025/0215422 A1
(43) Pub. Date: Jul. 3, 2025

(54) RT-DNA Fidelity and Retron Genome Editing

(71) Applicant: The J. David Gladstone Institutes, a testamentary trust established under the Will of J. David Glads, San Francisco, CA (US)

(72) Inventor: Seth Shipman, San Francisco, CA (US)

(21) Appl. No.: 18/850,957

(22) PCT Filed: Mar. 24, 2023
(86) PCT No.: PCT/US2023/016263

§ 371 Date: Sep. 25, 2024

Related U.S. Application Data
(60) Provisional application No. 63/323,536, filed on Mar. 25, 2022.

(51) Int. Cl. C12N 15/10; C12N 9/12; C12N 9/22; C12N 15/11; C12N 15/85; C12N 15/90

(52) U.S. Cl. CPC C12N 15/1082; C12N 9/1276; C12N 9/22; C12N 15/111; C12N 15/85; C12N 15/90; C12N 2310/20; C12Y 207/07049

(57) ABSTRACT

Described herein are compositions and methods that can analyze optimal systems for improved fidelity and frequency of genomic editing. The compositions and methods involve use of modified retrons.

### PRIORITY APPLICATION

This application claims the benefit of priority to U.S. Provisional Patent Application Ser. No. 63/323,536, filed Mar. 25, 2022, the content of which is incorporated herein by reference in its entirety.

### INCORPORATION BY REFERENCE OF SEQUENCE LISTING

A Sequence Listing is provided herewith as an xml file, “2319215.xml” created on Mar. 21, 2023, and having a size of 44,509 bytes. The content of the xml file is incorporated by reference herein in its entirety.

### BACKGROUND

Exogenous DNA can be introduced into cells as a template to edit the cell's genome. However, the amounts of exogenous DNA that can be introduced into cells are limited and not all cells will be transformed by the exogenous DNA. Moreover, it is often unclear which type of exogenous DNA can optimally edit a genomic site and which target site works best for editing.

Retron DNA is a useful source of such template DNA because retron DNA can be made abundantly in vivo by reverse transcription from retron RNA using the retron's own reverse transcriptase. However, genomic editing can still vary depending on the retron structures and sequences used, as well as the different genomic target sites that may be selected for modification.

### SUMMARY

Described herein are compositions and methods that can be used the evaluate which genomic editing systems provide optimal editing fidelity and frequency.

For example, the method can include: (a) transforming a population of host cells, each host cell comprising a reverse transcriptase and a cas nuclease, with the library of expression cassettes, each expression cassette comprising a promoter operably linked to a nucleic acid segment encoding a modified retron non-coding RNA (ncRNA) comprising a sequence for a barcode, a sequence for a donor DNA, and sequence for a guide RNA, and; (b) sequencing genomic sites comprising the barcodes within the host cells to determine (i) the identity and frequency of the barcodes in the population, (ii) the sequences of genomic edits made by the guide RNA and cas nuclease, or (iii) a combination thereof.

The methods and compositions described herein can be used to evaluate many variables of a genomic editing system, including different ncRNA chasses, different gRNA sequences, different gRNA designs, different reverse transcriptases used for generating the reverse transcribed DNA (donor DNAs), different CRISPR nucleases, and different genomic sites to be edited.

The guide RNA and the donor RNA can be encoded within the retron non-coding RNA region of the expression cassette. The barcode can be within the donor DNA to be a tag that uniquely identifies the presence of the donor DNA in a particular host cell genome. The barcode can be near a primer binding site that can be used to initiate sequencing of host cell genome and can be sequenced with or without other genomic sequences. The frequency of a particular bar code within a population of genomically edited host cells can be used as an indicator of the efficiency of genomic editing by a particular combination of genomic editing components.

The ncRNAs that can be employed can be modified versions of one or more types of retron (e.g., Eco1, Eco2, Ec48, E67, Ec73, Ec78, EC83, EC86, EC107, Ec107, Mx65, Mx162, Sa163, Vc81, Vc95, Vc137, Vc96, Ne144, or a combination thereof).

### DESCRIPTION OF THE FIGURES

FIG. 1A-1D illustrate some of the features of the constructs and methods described herein. FIG. 1A is a schematic of a construct that includes an expression cassette designed for synthesis of retron variants. The construct illustrated potential sites for segments that encode donor DNA (blue) and guide RNA (red) that can edit genomic sites, as well as a barcode (orange, e.g., within the donor DNA). Adaptors can be used to facilitate construction of a library and a tracrRNA can be encoded within the region that will be transcribed but, in this example, the tracrRNA segment is not within the cassette that encodes the donor DNA, guide RNA and barcode. In one experiment 3,125 variant donor DNAs were designed for evaluation of methods and constructs for editing at 25 different genomic sites. FIG. 1B illustrates structures of a series different variant ncRNAs that can be expressed from a construct such as the one illustrated in FIG. 1A. The different ncRNAs can include a one or more gRNAs. Upon expression, the ncRNAs are partially reversed transcribed (e.g., by the retron's own reverse transcriptase) to produce reverse transcribed DNA (RT-DNA) that provides multiple copies of the donor DNA for genomic editing. FIG. 1C illustrates targeting of the variant reverse transcribed retron DNA (RT-DNA, e.g., donor DNAs) to the same chromosomal site in different host cells. FIG. 1D is a schematic of a genomic site edited by a library of variant donor DNAs and gRNAs. As illustrated, the donor site in the genome can be evaluated by sequencing to determine which donor, gRNA, Cas nuclease, and other variables optimally edit that genomic site.

FIG. 2A-2G illustrate evaluation of ncRNA/RT-DNA features as well as retron similarities and differences. FIG. 2A shows a schematic of Eco1 and Eco4 ncRNAs, illustrating a difference between them in the loop identified as having positions 1-3. Both have a1/a2 and stem-loop regions that can be modified as described herein (the a1/a2 regions are labeled and the stem-loop regions are shown in blue). FIG. 2B illustrates the relative abundance of RT-DNA from Eco1 variants having modified loop bases at positions 1-3 of the loop shown in FIG. 2A. Deeper red shades indicate more RT-DNA production. As illustrated, use of thymine in the DNA encoding the ncRNA (before expression), or uracil in the ncRNA, at position 2 in the loop indicated in FIG. 2A of the Eco1 retron ncRNA provides improved RT-DNA (donor DNA) production. FIG. 2C illustrates the relative abundance of RT-DNA from Eco4 variants having modified loop bases at the positions indicated in FIG. 2A. Deeper red shades indicate more RT-DNA production. As illustrated, use of thymine in the DNA encoding the ncRNA, or uracil in the ncRNA, at position 2 in the loop indicated in FIG. 2A does not improve RT-DNA (donor DNA) production in the Eco4 ncRNA as much as it does for the Eco1 ncRNA. Eco4 is less vulnerable than Eco1 to sequence alterations in this loop. FIG. 2D graphically illustrates the relative RT-DNA abundance of each Eco1 stem length variant analyzed, where the RT-DNA abundance is shown as a percentage of wild-type abundance (dashed line). As illustrated, the RT-DNA abundance varies depending upon the length of the stem up to about stem length 15. FIG. 2E graphically illustrates the relative RT-DNA abundance of different Eco1 a1/a2 stem length variants as a percentage of wild-type abundance (dashed line). FIG. 2F graphically illustrates the relative RT-DNA abundance of each Eco4 stem length variant as a percentage of wild-type abundance (dashed line). FIG. 2G graphically illustrates the relative RT-DNA abundance of different Eco4 a1/a2 length variants as a percentage of wild-type abundance (dashed line).

FIG. 3A-3B illustrate modified retrons that can be used in genome editing. FIG. 3A shows a schematic of a ncRNA having a RT-DNA template for recombineering. The retron ncRNA was modified in the msd region (blue) to include a long loop (green) that contains a region encoding a DNA donor sequence with homology to a genomic locus, but where the DNA donor sequence this has one or more nucleotide modifications (repair nucleotides; asterisks). Such an ncRNA therefore provide a template for a donor DNA that is made by reverse transcription. FIG. 3B graphically illustrates fold enrichment of reverse transcribed DNA (RT-DNA) when an Eco1-based retron ncRNA has a longer stem (a1/a2) region of 22 nucleotides, compared to just 12 nucleotides. The RT-DNA products were detected qPCR, with the RT-DNA from each construct shown relative to uninduced. Circles show each of three biological replicates, with black for the wild type a1/a2 length and green for the extended a1/a2. This experiment was performed using procedures like those used for the data obtained for FIGS. 2E and 2G. See FIG. 2 for location of the retron a1/a2 region.

FIG. 4 illustrates some structural features of retrons that can be modified in a library of retrons.

FIG. 5A-5C. FIG. 5A provides relative editing of variants based on RT-Donor length. Small circles are individual variants, each normalized to a 94 length RT-Donor in sets of variants matched on all other parameters. Large circles are geometric mean of the variants. FIG. 5B provides relative editing of variants based on the RT-Donor and gRNA offset around the barcode insertion point (orange dotted line). FIG. 5C provides relative editing of variants based on ncRNA chassis modifications. Small circles are individual variants, each normalized to a reference chassis (Ref) that was the current standard Editron chassis prior to these experiments, in sets of variants matched on all other parameters. Large circles are geometric mean of the variants.

### DETAILED DESCRIPTION

Methods for optimizing variables that impact the fidelity and frequency of genomic editing are described herein. The methods described herein can include generating libraries of modified retron variants and evaluating the results of genomic editing by the different modified retron variants. A variety of variables can be evaluated, including different ncRNA chasses, different encoded gRNA sequences, different gRNA designs, different reverse transcriptases used for generating the variant retron RT-DNA, different CRISPR nucleases, and different genomic sites to be edited. The amounts of reverse transcribed retron DNA (RT-DNA) from an ncRNA template need not be quantified. Each type of donor RT-DNA can include a unique bar code to facilitate analysis of genomic editing frequency and editing fidelity at a genomic site.

Constructs are generated that can express the different ncRNAs, gRNA sequences, gRNA designs, reverse transcriptases, and retron structures used for generating the variant retron DNA, CRISPR nucleases, or combinations thereof. Such constructs can be linked to a barcode. The linked bar code is inserted into the genome along with retron variant DNA, retron reverse transcriptases, CRISPR nucleases, or combinations thereof. Segments of DNA with the bar code can be recovered and evaluated by sequencing. In some cases, the relative integration frequency of genomic edits can be assessed. In this way, many variables can be evaluated in the same population of cells. Hence, the variables relating to the fidelity and relative integration frequency of genomic edits can be identified and the genomic editing frequencies and fidelities can be optimized.

The effects of the different variables can also be evaluated in different host cells. Natural genomic sites as well as engineered genomic sites can be used as target insertion sites. When such engineered genomic sites are identical in different host cells, the effects of other variables and the efficiency of modification in the different species can be evaluated.

Such experiments facilitate identification of superior methods and constructs for genomic editing and also improve our understanding of human genomic editing and editing in other species. These experiments allow optimal design of the components that do the editing as well as identification of optimal genomic sites for editing. Hence, the methods and compositions described herein facilitate design of improved ncRNA chassis, gRNA sequences, gRNA designs, reverse transcriptases, CRISPR nucleases, and combinations thereof.

Libraries of modified retron variants for genomic editing can be generated, tested, and evaluated for their effects editing. The amounts of reverse transcribed retron DNA (RT-DNA) from an ncRNA template may or may not be quantified. Instead, the frequency and fidelity of editing at genomic sites are quantified.

The variables to be evaluated can include the ncRNA chassis, gRNA sequence, the gRNA design, the reverse transcriptase (RT), the CRISPR nuclease, and the genomic editing site itself.

### Retrons

Methods are described herein for analyzing the efficiency, fidelity, and frequency of genomic editing by retron nucleic acids, including libraries of retron nucleic acids, retron variants, retron mutants, engineered retrons, or combinations thereof. The retron nucleic acids can also be modified to include useful exogenous or heterologous nucleic acids, thereby allowing production in vivo of substantial amounts of products such as gRNAs, templates for genomic repair, templates for reverse transcriptases, and the like.

Retrons in nature generally include two elements, one that encodes a reverse transcriptase and a second that is single-stranded DNA/RNA hybrid called a multicopy single-stranded DNA (msDNA). Wild type retrons are about 2 kb long. They contain a single operon controlling the synthesis of an RNA transcript carrying three loci, msr, msd, and ret, that are involved in msDNA synthesis. The DNA portion of msDNA is encoded by the msd gene, the RNA portion is encoded by the msr gene, while the product of the ret gene is a reverse transcriptase. The retron msr RNA is a non-coding RNA (ncRNA) produced by retron elements and is the immediate precursor to the synthesis of msDNA.

While msDNA and reverse transcribed DNA (RT-DNA) are related, the term reverse transcribed DNA (RT-DNA) is used herein to refer to any retron-related reverse transcribed DNA, whether modified or not, while the term msDNA refers to wild type, natural, or unmodified retron msDNA.

The ncRNA of naturally occurring retrons includes a pre-msr sequence, an msr gene encoding multicopy single-stranded RNA (msRNA). The msd gene encodes a multicopy single-stranded DNA (msDNA), the post-msd sequence, and a ret gene encoding a reverse transcriptase. Synthesis of DNA by the retron-encoded reverse transcriptase provides a DNA/RNA chimeric product which is composed of single-stranded DNA encoded by the msd gene linked to single-stranded RNA encoded by the msr gene. The retron msr RNA contains a conserved guanosine residue at the end of a stem loop structure. A strand of the msr RNA is joined to the 5′ end of the msd single-stranded DNA by a 2′-5′ phosphodiester linkage at the 2′ position of this conserved guanosine residue.

For example, a wild type retron-Eco1 ncRNA (also called ec86 or retron-Eco1 ncRNA) can have the sequence shown below as SEQ ID NO:1.

```
    1 TGCGCACCCT TAGCGAGAGG TTTATCATTA AGGTCAACCT
  41 CTGGATGTTG TTTCGGCATC CTGCATTGAA TCTGAGTTAC
  81 TGTCTGTTTT CCTTGTTGGA ACGGAGAGCA TCGCCTGATG
121 CTCTCCGAGC CAACCAGGAA ACCCGTTTTT TCTGACGTAA
161 GGGTGCGCA
```

An example of an Eco1 human-codon optimized reverse transcriptase (RT) sequence that can be used is shown below as SEQ ID NO:2.

```
    1 ATGAAATCTG CAGAGTATCT GAATACGTTC CGCCTTAGGA
  41 ATTTGGGCCT CCCCGTGATG AACAATCTCC ACGATATGAG
  81 CAAGGCGACT CGAATATCCG TGGAAACGCT GAGACTGCTC
121 ATCTATACAG CAGACTTTCG GTACAGGATC TACACGGTCG
161 AAAAGAAGGG GCCTGAGAAA CGCATGCGAA CAATTTATCA
201 ACCTAGCCGA GAGCTCAAGG CGTTGCAGGG CTGGGTTCTT
241 CGAAACATCC TTGACAAACT CTCATCATCA CCCTTTAGTA
281 TTGGGTTTGA AAAGCACCAA AGCATCCTTA ACAACGCGAC
321 GCCACACATA GGTGCCAATT TCATATTGAA CATCGACTTG
361 GAGGATTTTT TTCCGAGCCT CACAGCCAAT AAAGTGTTCG
401 GTGTTTTTCA CAGTCTTGGG TACAATCGCC TTATTAGTTC
411 CGTTCTTACC AAGATTTGTT GTTACAAGAA TCTCTTGCCC
481 CAGGGAGCAC CCAGCAGTCC GAAATTGGCG AATTTGATTT
521 GTTCCAAGCT CGATTATCGA ATACAAGGGT ACGCGGGCAG
561 CCGGGGACTC ATCTATACCC GCTACGCAGA CGATCTTACG
601 CTGTCTGCCC AATCAATGAA GAAGGTCGTA AAGGCGCGGG
641 ATTTCTTGTT TTCTATCATC CCGTCCGAGG GCTTGGTAAT
681 TAATTCCAAA AAGACTTGTA TCTCAGGACC ACGATCTCAG
721 CGAAAAGTGA CAGGACTCGT CATTTCTCAA GAAAAAGTCG
761 GTATAGGGAG AGAGAAGTAT AAGGAAATCC GCGCGAAGAT
801 CCACCACATA TTCTGTGGCA AGAGCAGCGA GATAGAACAC
841 GTCCGAGGCT GGTTGTCCTT CATACTGAGC GTGGACTCAA
881 AAAGCCACCG CCGGTTGATC ACCTATATTT CAAAACTGGA
921 AAAGAAATAT GGAAAGAACC CACTCAACAA AGCTAAAACA
961 TAG
```

An example of an Eco2 human-codon optimized reverse transcriptase (RT) sequence is shown below as SEQ ID NO:3.

```
      1 ATGACAAAAA CTTCAAAGCT GGATGCGCTG CGGGCGGCTA
    41 CTAGTAGGGA AGATTTGGCG AAGATTCTCG ACATAAAGTT
    81 GGTGTTTCTG ACAAACGTGT TGTACCGCAT AGGATCCGAC
  121 AACCAGTATA CGCAATTCAC AATACCCAAA AAGGGTAAAG
  161 GTGTCCGCAC CATCAGCGCA CCAACGGACC GACTTAAGGA
  201 TATACAGAGG AGGATTTGTG ATCTTCTTAG TGACTGTAGG
  241 GATGAAATCT TTGCGATTAG GAAGATCTCT AATAATTACT
  281 CATTCGGCTT CGAAAGAGGA AAATCAATTA TACTCAATGC
  321 TTACAAGCAT CGAGGGAAGC AAATTATATT GAACATCGAC
  361 CTTAAGGACT TCTTTGAGAG CTTTAACTTT GGGAGAGTCC
  401 GGGGGTACTT TCTCTCCAAC CAGGACTTCT TGTTGAACCC
  441 AGTIGTGGCA ACAACGTTGG CGAAGGCCGC CTGCTACAAC
  481 GGGACTCTGC CTCAGGGGTC CCCATGTTCC CCTATTATAA
  521 GTAACCTTAT CTGTAACATT ATGGACATGC GGCTCGCAAA
  561 GCTCGCCAAG AAGTACGGCT GCACTTATAG TCGATATGCG
  601 GATGACATTA CGATCAGCAC CAATAAAAAT ACCTTCCCGT
  641 TGGAGATGGC GACTGTGCAG CCTGAAGGGG TTGTGCTGGG
  681 CAAAGTGCTC GTAAAGGAGA TTGAAAATTC AGGTTTCGAG
  721 ATTAACGATT CTAAGACTAG ATTGACCTAC AAAACAAGTA
  761 GGCAAGAAGT CACCGGGCTG ACGGTTAATC GGATTGTAAA
  801 CATTGATCGG TGCTACTACA AAAAGACGAG GGCGCTGGCT
  841 CACGCATTGT ATCGGACAGG AGAATATAAG GTCCCAGACG
  881 AGAACGGTGT TCTGGTATCT GGAGGGCTTG ACAAGTTGGA
  921 GGGTATGTTT GGGTTTATCG ACCAGGTGGA TAAATTCAAC
  961 AACATTAAAA AAAAGTTGAA TAAGCAACCC GACAGATATG
1001 TTCTGACAAA TGCCACTTTG CACGGATTTA AGCTCAAATT
1041 GAACGCCAGG GAGAAAGCCT ATAGCAAATT CATCTACTAC
1081 AAATTCTTCC ACGGTAATAC TTGTCCCACG ATCATAACAG
1121 AGGGTAAGAC GGATAGGATT TACCTTAAAG CTGCCCTCCA
1161 TAGCCTCGAG ACAAGTTATC CTGAACTGTT TCGGGAGAAA
1201 ACAGATAGTA AGAAGAAGGA GATAAATCTG AATATTTTTA
1241 AAAGCAATGA GAAGACCAAG TATTTCCTGG ATCTCAGCGG
1281 CGGCACAGCA GACCTCAAGA AATTCGTGGA ACGCTACAAA
1321 AATAACTACG CTTCCTATTA CGGCAGCGTA CCGAAACAAC
1361 CGGTGATAAT GGTGCTTGAT AACGACACAG GCCCGTCAGA
1401 CCTGTTGAAC TTTTTGAGAA ACAAAGTTAA GAGTTGTCCA
1441 GATGATGTAA CAGAAATGCG CAAGATGAAG TACATACATG
1481 TGTTTTACAA TCTGTACATA GTTCTGACTC CCCTGTCTCC
1521 ATCTGGAGAG CAAACGTCTA TGGAGGACCT CTTTCCTAAA
1561 GATATATTGG ACATTAAGAT AGATGGCAAG AAATTCAATA
1601 AAAACAATGA CGGTGACTCC AAAACAGAGT ATGGGAAGCA
1001 CATATTCTCA ATGCGCGTTG TACGAGATAA AAAGAGGAAG
1001 ATAGATTTCA AGGCATTTTG CTGTATCTTC GATGCTATTA
1001 AGGATATTAA AGAACATTAC AAACTGATGT TGAATTCCTA
1001 G
```

An example of an Eco1 wild-type retron reverse transcriptase sequence is shown below as SEQ ID NO:4.

```
    1 KSAEYLNTER LRNLGLPVMN NLHDMSKATR ISVETLRLLI
  41 YTADFRYRIY TVEKKGPEKR MRTIYQPSRE LKALQGWVLR
  81 NILDKLSSSP FSIGFEKHQS ILNNATPHIG ANFILNIDLE
121 DFFPSLTANK VFGVFHSLGY NRLISSVLTK ICCYKNLLPQ
161 GAPSSPKLAN LICSKLDYRI QGYAGSRGLI YTRYADDLTL
201 SAQSMKKVVK ARDFLFSIIP SEGLVINSKK TCISGPRSQR
241 KVTGLVISQE KVGIGREKYK EIRAKIHHIF CGKSSEIEHV
281 RGWLSFILSV DSKSHRRLIT YISKLEKKYG KNPLNKAKT
```

An example of an Eco2 wild-type retron reverse transcriptase sequence is shown below as SEQ ID NO:5.

```
    1 MTKTSKLDAL RAATSREDLA KILDIKLVFL TNVLYRIGSD
  41 NQYTQFTIPK KGKGVRTISA PTDRLKDIQR RICDLLSDCR
  81 DEIFAIRKIS NNYSFGFERG KSIILNAYKH RGKQIILNID
121 LKDFFESENF GRVRGYFLSN QDFLLNPVVA TTLAKAACYN
161 GTLPQGSPCS PIISNLICNI MDMRLAKLAK KYGCTYSRYA
201 DDITISTNKN TFPLEMATVQ PEGVVLGKVL VKEIENSGFE
241 INDSKTRLTY KTSRQEVTGL TVNRIVNIDR CYYKKTRALA
281 HALYRTGEYK VPDENGVLVS GGLDKLEGMF GFIDQVDKEN
321 NIKKKLNKQP DRYVLTNATL HGFKLKLNAR EKAYSKFIYY
361 KFFHGNTCPT IITEGKTDRI YLKAALHSLE TSYPELFREK
401 TDSKKKEINL NIFKSNEKTK YFLDLSGGTA DLKKEVERYK
441 NNYASYYGSV PKQPVIMVLD NDTGPSDLLN FLRNKVKSCP
481 DDVTEMRKMK YIHVFYNLYI VLTPLSPSGE QTSMEDLEPK
521 DILDIKIDGK KENKNNDGDS KTEYGKHIFS MRVVRDKKRK
561 IDFKAFCCIF DAIKDIKEHY KLMLNS
```

An example of a sequence for an Eco4 retron reverse transcriptase is shown below as SEQ ID NO:6.

```
    1 MSIDIETTLQ KAYPDFDVLL KSRPATHYKV YKIPKRTIGY
  41 RIIAQPTPRV KAIQRDIIEI LKQHTHIHDA ATAYVDGKNI
  81 LDNAKIHQSS VYLLKLDLVN FENKITPELL FKALARQKVD
121 ISDTNKNLLK QFCFWNRTKR KNGALVLSVG APSSPFISNI
161 VMSSFDEEIS SFCKENKISY SRYADDLTES TNERDVLGLA
201 HQKVKTTLIR FFGTRIIINN NKIVYSSKAH NRHVTGVTLT
241 NNNKLSLGRE RKRYITSLVF KFKEGKLSNV DINHLRGLIG
281 FAYNIEPAFI ERLEKKYGES TIKSIKKYSE GG
```

An example of a sequence for a Sen2 retron reverse transcriptase is shown below as SEQ ID NO:7.

```
    1 MDILQHISDL LLTKKSEIIS FSLTAPYRYK IYKIAKRNSD
  41 KKRTIAHPSK ELKFIQREIT EYLTDKLPVH ECAFAYKKGS
  81 SIKTNAQVHL HTKYLLKMDF ENFFPSITPR LFFSKLRLAN
121 IDLTADDKVL LENILFFKSK RNSNLRLSIG APSSPLISNE
161 VMYFWDIEVQ EICSKIGVNY TRYADDLTFS TNNKDVLFDI
201 PDMLENVLPK YSLGRIRINH EKTVFSSKGH NRHVTGITLT
241 NDNKLSIGRE RKRKISAMIH HFINGKLSTD ECNKLVGLLA
281 FAKNIEPSFY KSMVIKYGSD NIYKLQKQKD K
```

Other types of retrons are described throughout the application and can be used in the methods described herein.

Modified (e.g., engineered) retrons can have alterations in different locations relative to the corresponding wild type retrons. However, not every modification provides a stable retron or one that can yield good amounts of reverse transcribed DNA. Hence, the methods described herein provide procedures for identifying which modifications are optimal for obtaining desired results.

One example of a location for modification of retron nucleic acids is within a self-complementary region (stem region, which has sequence complementarity to the pre-msr sequence), wherein the length of the self-complementary region can be lengthened relative to the corresponding region of a native retron. Complementarity between the strands of the stem region is maintained but the length of the stem region can be increased. Such modifications result in an engineered retron that provides enhanced production of RT-DNA.

In certain embodiments, the complementary region has a length at least 1, at least 2, at least 4, at least 6, at least 8, at least 10, at least 12, at least 14, at least 16, at least 18, at least 20, at least 30, at least 40, or at least 50 nucleotides longer than the wild-type self-complementary region. For example, the self-complementary region may have a length ranging from at least 1 to at least 50 nucleotides longer than the native or wild-type complementary region, including any length within this range, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 34, 35, 36, 37, 38, 39, 40, 41, 42, 43, 44, 45, 46, 47, 48, 49, 50 or more nucleotides longer. Such modifications should retain the complementarity of the stem structure. In certain embodiments, the self-complementary region has a length ranging from 1 to 16 nucleotides longer than the wild-type complementary region.

To create more abundant RT-DNA, for example, the ncRNA SEQ ID NO:8 sequence shown below, with the native self-complementary 3′ and 5′ ends highlighted in bold (at positions 1-12 and 158-169), can be extended at positions 1 and 169 to extend the self-complementary region.

```
    1 TGCGCACCCT TAGCGAGAGG TTTATCATTA AGGTCAACCT
  41 CTGGATGTTG TTTCGGCATC CTGCATTGAA TCTGAGTTAC
  81 TGTCTGTTTT CCTTGTTGGA ACGGAGAGCA TCGCCTGATG
121 CTCTCCGAGC CAACCAGGAA ACCCGTTTTT TCTGACGTAA
161 GGGTGCGCA
```

For example, as shown below for the following engineered “ncRNA extended” (SEQ ID NO: 9) construct, where the additional nucleotides that extend the self-complementary region are shown in italics with underlining.

```
    1 TGATAAGATT CCGTATGCGC ACCCTTAGCG AGAGGTTTAT
  41 CATTAAGGTC AACCTCTGGA TGTTGTTTCG GCATCCTGCA
  81 TTGAATCTGA GTTACTGTCT GTTTTCCTTG TTGGAACGGA
121 GAGCATCGCC TGATGCTCTC CGAGCCAACC AGGAAACCCG
161 TTTTTTCTGA CGTAAGGGTG CGCATACGGA ATCTTATCA
```

In some cases, the additional nucleotides can be added to any position in the self-complementary region, for example, anywhere within positions 1-12 and 158-169 of the SEQ ID NO: 8 or SEQ ID NO:9 sequence.

In certain embodiments, sequences of the msr gene, msd gene, and ret gene used in the engineered retron may be derived from any bacterial retron operon. Representative retrons are available such as those from gram-negative bacteria including, without limitation, myxobacteria retrons such as Myxococcus xanthus retrons (e.g., Mx65, Mx162) and Stigmatella aurantiaca retrons (e.g., Sa163); Escherichia coli retrons (e.g., Ec48, E67, Ec73, Ec78, EC83, EC86, EC107, and Ec107); Salmonella enterica, Vibrio cholerae retrons (e.g., Vc81, Vc95, Vc137); Vibrio parahaemolyticus (e.g., Vc96); and Nannocystis exedens retrons (e.g., Ne144). Retron msr gene, msd gene, and ret gene nucleic acid sequences as well as retron reverse transcriptase protein sequences may be derived from any source. Representative retron sequences, including msr gene, msd gene, and ret gene nucleic acid sequences and reverse transcriptase protein sequences are listed in the National Center for Biotechnology Information (NCBI) database. See, for example, NCBI entries: Accession Nos. EF428983, M55249, EU250030, X60206, X62583, AB299445, AB436696, AB436695, M86352, M30609, M24392, AF427793, AQ3354, and AB079134; all of which sequences (as entered by the date of filing of this application) are herein incorporated by reference in their entireties.

The retron ncRNAs can be modified to enhance production of retron reverse transcribed DNA in a host cell or to provide host cells with genomic editing components or other useful proteins and/or nucleic acids. Any of the foregoing retron sequences (or variants thereof) can include variant or mutant nucleotides, added nucleotides, or fewer nucleotides.

For example, a parental ncRNA can be modified by addition of nucleotides to a stem or loop as described herein. Before modification the parental ncRNA can have at least about 80-100% sequence identity to any region of the retrons described herein, including any percent identity within this range, including any percent identity within this range, such as 81, 82, 83, 84, 85, 86, 87, 88, 89, 90, 91, 92, 93, 94, 95, 96, 97, 98, or 99% sequence identity to any region of the retron sequences described herein (including those defined by accession number). Such parental retrons can be used to construct an engineered retron or vector system comprising an engineered retron, as described herein.

The variant retrons can include exogenous or heterologous nucleotides or nucleic acid segments. For example, the exogenous or heterologous nucleotide or nucleic acid segments can add at least 1, at least 2, at least 4, at least 6, at least 8, at least 10, at least 12, at least 14, at least 16, at least 18, at least 20, at least 30, at least 40, at least 50, at least 60, at least 70, at least 80, at least 90, at least 100, at least 125, at least 150, at least 175, or at least 200 nucleotides to parental retron nucleic acids, to thereby generate variant retron nucleic acids.

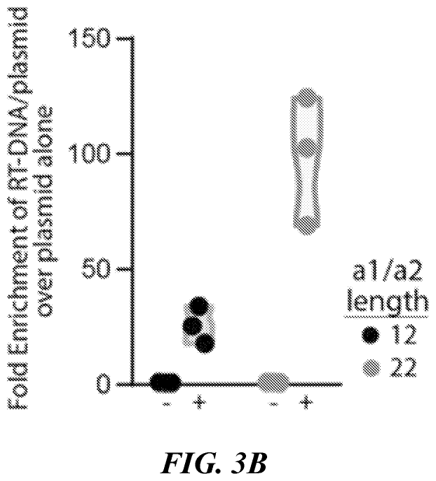

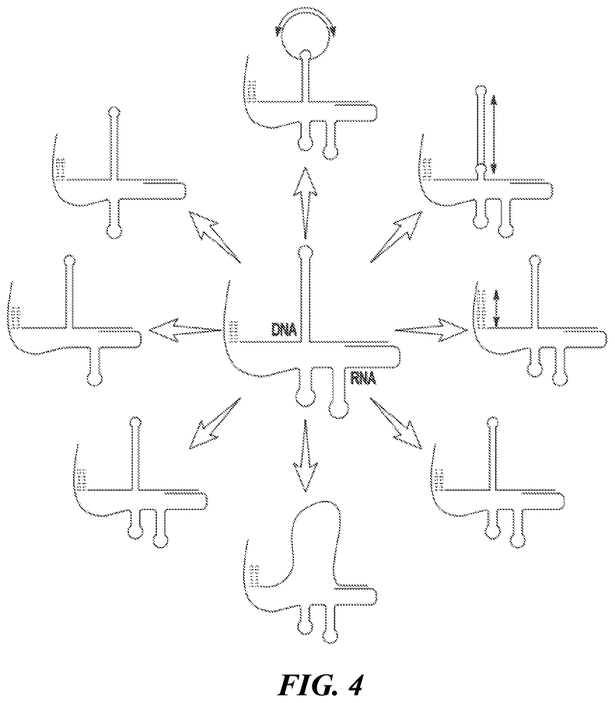

One example of a locus for insertion of exogenous or heterologous nucleotide or nucleic acid segments into retron nucleic acids is a loop portion of a stem-loop (see, e.g., FIG. 3A).

As described above, the retron nucleic acids can be modified with respect to the native retron to include one or more heterologous sequences of interest, including a donor polynucleotide suitable for use in gene editing, e.g., by homology directed repair (HDR) or recombination-mediated genetic engineering (recombineering), a barcode, a guide RNA (e.g., with the tracrRNA), as discussed further below. Such heterologous sequences may be inserted, for example, into the ncRNA coding region in the expression cassette. Upon transcription, the ncRNA will contain the guide RNA, as well as the RNA segment encoding the donor DNA. The ncRNA can be partially reverse transcribed to generate the donor RNA.

In some cases, the donor DNA sequence of interest can be inserted into the loop of the msd stem loop of the retron.

In some cases, engineered retron nucleic acids can include unique barcodes to facilitate multiplexing. Barcodes may comprise one or more nucleotide sequences that are used to identify a nucleic acid or cell with which the barcode is associated. Such barcodes may be inserted for example, into the loop region of the msd-encoded DNA. Barcodes can be 3-1000 or more nucleotides in length, preferably 10-250 nucleotides in length, and more preferably 10-30 nucleotides in length, including any length within these ranges, such as 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, or 1000 nucleotides in length. A barcode may be used to identify the presence of a particular genetically modified site within a host cell. The use of barcodes allows retrons from different cells to be pooled in a single reaction mixture for sequencing while still being able to trace a particular retron, ncRNA, donor DNA, reverse transcriptase, or cas nuclease back to the colony from which it originated.

Therefore, expression cassettes with segments encoding any of the ncRNAs, donor DNAs, guide RNAs, reverse transcriptases, and/or cas nucleases can be linked to a barcode that is inserted into the genome and can be recovered by sequencing. In this way, many variables can be identified and evaluated in the same population of cells to assess relative integration frequency. One embodiment provides an expression cassette comprising a promoter operably linked to a nucleic acid segment encoding a modified retron ncRNA comprising a sequence for a barcode, a sequence for a donor DNA, and sequence for a guide RNA. In one embodiment, the barcode is within the sequence for the donor DNA. In another embodiment, each barcode is a marker identifying a particular donor DNA. In one embodiment, each barcode is about 10 to 250 nucleotides in length. In another embodiment, the sequence for the donor DNA comprises one or more variant nucleotides compared to a genomic DNA target site sequence. In one embodiment, each guide RNA recognizes and can bind to a genomic DNA gRNA binding site within 100 to 1000 nucleotides of a genomic DNA target site to be edited. In one embodiment, the expression cassette further comprises at least one or two adapters, restriction sites, or a combination thereof. In another embodiment, expression cassette further encodes a trans-activating crRNA (tracrRNA). In one embodiment, each barcode is a marker identifying a specific ncRNA variant. In some embodiments, barcodes provided herein are associated with editing donors and are linked/associated with linked to variations in the retron ncRNA, including nucleotide mutations, insertions, and deletions.

The modified retron constructs can have a non-native configurations with non-native spacing between the ncRNA coding region and the reverse transcriptase (ret) coding region. For example, it can be useful to separate the expression cassettes that include the ncRNA coding region and the reverse transcriptase (ret) coding region. Hence, the ncRNA and the reverse transcriptase may be separated in a trans arrangement rather than provided in the natural cis arrangement. In some embodiments, the ret gene is provided in a trans arrangement that eliminates a cryptic stop signal for the reverse transcriptase, which allows the generation of longer single stranded DNAs from the engineered retron construct.

Amplification of retron nucleic acids may be performed, for example, before introduction into cells, before ligation into vectors, or at other times. Any method for amplifying the retron constructs may be used, including, but not limited to polymerase chain reaction (PCR), isothermal amplification, nucleic acid sequence-based amplification (NASBA), transcription mediated amplification (TMA), strand displacement amplification (SDA), and ligase chain reaction (LCR). In one embodiment, the retron constructs comprise common 5′ and 3′ priming sites to allow amplification of retron sequences in parallel with a set of universal primers. In another embodiment, a set of selective primers is used to selectively amplify a subset of retron sequences from a pooled mixture.

The methods can be performed in a variety of host cell types, within naturally occurring genomic sites or engineered sites of such cells. For example, engineered genomic sites can be inserted into host cells to reduce variability that may be present in related but non-identical genomic sites across different host cell types. When such engineered genomic sites are identical in different species, for example, the effects of other variables and the efficiency of modification in the different species can be evaluated. Such experiments facilitate understanding of human editing and editing in other species.

The methods and compositions therefore allow optimal design of the editing sites as well as design of improved ncRNA chassis, gRNA sequences, gRNA designs, reverse transcriptases, CRISPR nucleases, and combinations thereof.

### Libraries

Libraries of modified nucleic acids (modified ncRNAs, modified DNAs encoding ncRNAs, modified DNAs encoding reverse transcriptases, DNAs encoding different guide RNAs, DNAs encoding different cas nucleases, and combinations thereof) can be used in the expression cassettes, constructs and methods described herein. Thousands of nucleic acids encoding modified retron ncRNAs, different reverse transcriptases, various guide RNAs, different cas nucleases, and combinations thereof can be synthesized to systematically test each variable of the genomic editing system.

A golden-gate-based cloning strategy (Engler et al., PLOS One (Nov. 5, 2008)) can be used to clone such nucleic acids, and then large pools of modified retron ncRNAs, different reverse transcriptases, various guide RNAs, different cas nucleases, and combinations thereof can be expressed in multiplexed vectors.

For example, a plasmid having or encoding a parental ncRNA nucleic acid insert (e.g., one that encodes a donor DNA and/or a guide RNA) can be subjected to directed mutagenesis to generate a population of plasmids with different nucleic acid inserts that encode the differently modified ncRNAs. The plasmid can be an expression vector or an expression cassette so that the nucleic acid inserts can be expressed to generate the different modified retron ncRNAs, along with the one or more reverse transcriptases, guide RNAs, cas nucleases, and combinations thereof.

Alternatively, a population of oligonucleotides encoding ncRNAs (e.g., one that encodes a donor DNA and/or a guide RNA) can be subjected to directed mutagenesis to generate a population of variant oligonucleotides, which can be inserted into expression vectors or expression cassettes so that the oligonucleotide inserts can be expressed to generate the variant ncRNAs, that can provide the donor DNAs and/or a guide RNAs. Genomic editing results that occur in host cells expressing a reverse transcriptase and a cas nuclease can be evaluated using the methods described herein.

### Retron Expression Systems

Modified and unmodified retrons, retron nucleic acids, ncRNAs, or retron constructs can be incorporated into and expressed from an expression cassette or expression vector. In general, the selected retron nucleic acids are one or more wild type or variant ncRNA, retron reverse transcriptases, as well as libraries or populations thereof. The retrons or retron libraries can be expressed from expression cassettes or expression vectors that can be present in vitro or in vivo within host cells.

Mutant, modified, or wild type retron ncRNAs, msr genes, msd genes, and/or ret genes can individually or collectively be expressed in vivo from an expression cassette or expression vector within a cell.

A “vector” is a composition of matter that can be used to deliver a nucleic acid of interest to the interior of a cell. Retron (modified and/or unmodified) nucleic acids can be introduced into a cell with a single vector or in multiple separate vectors to produce wild type, mutant or modified retron RNA (ncRNA) and/or DNA and/or reverse transcriptases in host cells. Vectors typically include control elements operably linked to the retron sequences, which allow for expression in vivo in the host cells. For example, the segment encoding the retron ncRNA and/or the segment encoding the ret (reverse transcriptase) can be operably linked to the same or different promoters to allow expression of the retron ncRNA, retron RT-DNA, and/or the retron reverse transcriptase.

In some embodiments, heterologous sequences encoding desired products of interest (e.g., guide RNAs, donor polynucleotides for gene editing, barcodes, or combinations thereof) may be inserted in the segment encoding the ncRNA.

Any eukaryotic, archeon, or prokaryotic cell, capable of being transfected with a vector comprising the engineered retron sequences, may be used as host cells for the retron-related expression cassettes and expression vectors. The ability of constructs to express ncRNA, RT-DNA, or other retron-encoded products (e.g., reverse transcriptases) can be empirically determined using the methods described herein.

In some embodiments, the engineered retron nucleic acids are produced by a vector system comprising one or more vectors. In the vector system, the ncRNA and the reverse transcriptase may be provided by the same vector (i.e., cis arrangement of such retron elements), wherein the vector comprises a promoter operably linked to the segment encoding the ncRNA and the segment encoding the reverse transcriptase. In some embodiments, a second promoter is operably linked to the segment encoding the reverse transcriptase. Alternatively, the segment encoding the reverse transcriptase may be incorporated into a second vector that does not include the ncRNA, msr gene or the msd gene (i.e., trans arrangement).

Numerous vectors are available including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, lentiviral vectors, and the like. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In certain embodiments, the nucleic acid comprising one or more wild type or modified retron sequences is under transcriptional control of a promoter. A “promoter” refers to a DNA sequence recognized by the synthetic machinery of the cell, or introduced synthetic machinery, required to initiate the specific transcription of a gene. The term promoter will be used here to refer to a group of transcriptional control modules that are clustered around the initiation site for RNA polymerase I, II, or III. Typical promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. These and other promoters can be obtained from commercially available plasmids, using techniques well known in the art. See, e.g., Sambrook et al., supra. Enhancer elements may be used in association with the promoter to increase expression levels of the constructs. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Expression vectors for expressing one or more retron nucleic acids can include a promoter “operably linked” to a nucleic acid segment encoding the ncRNA and/or the reverse transcriptase. The phrase “operably linked” or “under transcriptional control” as used herein means that the promoter is in the correct location and orientation in relation to a polynucleotide to control the initiation of transcription by RNA polymerase and expression of the ncRNA and/or the reverse transcriptase.

Typically, transcription terminator/polyadenylation signals will also be present in the expression construct. Examples of such sequences include, but are not limited to, those derived from SV40, as described in Sambrook et al., supra, as well as a bovine growth hormone terminator sequence (see, e.g., U.S. Pat. No. 5,122,458). Additionally, 5′-UTR sequences can be placed adjacent to the coding sequence in order to enhance expression of the same. Such sequences may include UTRs comprising an internal ribosome entry site (IRES).

Inclusion of an IRES permits the translation of one or more open reading frames from a vector. Such an IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20:102-110; Kobayashi et al., BioTechniques (1996) 21:399-402; and Mosser et al., BioTechniques (1997 22:150-161. A multitude of IRES sequences are available and include sequences derived from a wide variety of viruses, such as from leader sequences of picornaviruses such as the encephalomyocarditis virus (EMCV) UTR (Jang et al. J. Virol. (1989) 63:1651-1660), the polio leader sequence, the hepatitis A virus leader, the hepatitis C virus IRES, human rhinovirus type 2 IRES (Dobrikova et al., Proc. Natl. Acad. Sci. (2003) 100 (25): 15125-15130), an IRES element from the foot and mouth disease virus (Ramesh et al., Nucl. Acid Res. (1996) 24:2697-2700), a giardiavirus IRES (Garlapati et al., J. Biol. Chem. (2004) 279 (5): 3389-3397), and the like. A variety of nonviral IRES sequences will also find use herein, including, but not limited to IRES sequences from yeast, as well as the human angiotensin II type 1 receptor IRES (Martin et al., Mol. Cell Endocrinol. (2003) 212:51-61), fibroblast growth factor IRESs (FGF-1 IRES and FGF-2 IRES, Martineau et al. (2004) Mol. Cell. Biol. 24 (17): 7622-7635), vascular endothelial growth factor IRES (Baranick et al. (2008) Proc. Natl. Acad. Sci. U.S.A. 105 (12): 4733-4738, Stein et al. (1998) Mol. Cell. Biol. 18 (6): 3112-3119, Bert et al. (2006) RNA 12 (6): 1074-1083), and insulin-like growth factor 2 IRES (Pedersen et al. (2002) Biochem. J. 363 (Pt 1): 37-44). These elements are readily commercially available in plasmids sold, e.g., by Clontech (Mountain View, CA), Invivogen (San Diego, CA), Addgene (Cambridge, MA) and GeneCopoeia (Rockville, MD). See also IRESite: The database of experimentally verified IRES structures (iresite.org). An IRES sequence may be included in a vector, for example, to express a reverse transcriptase or an RNA-guided nuclease (e.g., Cas9) from an expression cassette.

Alternatively, a polynucleotide encoding a viral 2A-self cleaving peptide can be used to allow production of multiple protein products (e.g., Cas9, bacteriophage recombination proteins, retron reverse transcriptase) from a single vector. One or more 2A linker peptides can be inserted between the coding sequences in the multicistronic construct. The 2A peptide, which is self-cleaving, allows co-expressed proteins from the multicistronic construct to be produced at equimolar levels. 2A peptides from various viruses may be used, including, but not limited to 2A peptides derived from the foot-and-mouth disease virus, equine rhinitis A virus, Thosea asigna virus and porcine teschovirus-1. See, e.g., Kim et al. (2011) PLOS One 6 (4): e18556, Trichas et al. (2008) BMC Biol. 6:40, Provost et al. (2007) Genesis 45 (10): 625-629, Furler et al. (2001) Gene Ther. 8 (11): 864-873; herein incorporated by reference in their entireties.

In certain embodiments, the expression construct comprises a plasmid sequences suitable for transforming a bacterial host. Numerous bacterial expression vectors are available. Bacterial expression vectors include, but are not limited to, pACYC177, pASK75, pBAD, pBADM, pBAT, pCal, pET, pETM, pGAT, pGEX, pHAT, pKK223, pMal, pProEx, pQE, and pZA31. Bacterial plasmids may contain antibiotic selection markers (e.g., ampicillin, kanamycin, erythromycin, carbenicillin, streptomycin, or tetracycline resistance), a lacZ gene (β-galactosidase produces blue pigment from x-gal substrate), fluorescent markers (e.g., GFP. mCherry), or other markers for selection of transformed bacteria. See, e.g., Sambrook et al., supra.

In other embodiments, the expression construct comprises a plasmid suitable for transforming a yeast cell. Yeast expression plasmids typically contain a yeast-specific origin of replication (ORI) and nutritional selection markers (e.g., HIS3, URA3, LYS2, LEU2, TRP1, MET15, ura4+, leu1+, ade6+), antibiotic selection markers (e.g., kanamycin resistance), fluorescent markers (e.g., mCherry), or other markers for selection of transformed yeast cells. The yeast plasmid may further contain components to allow shuttling between a bacterial host (e.g., E. coli) and yeast cells. A number of different types of yeast plasmids are available including yeast integrating plasmids (YIp), which lack an ORI and are integrated into host chromosomes by homologous recombination; yeast replicating plasmids (YRp), which contain an autonomously replicating sequence (ARS) and can replicate independently; yeast centromere plasmids (YCp), which are low copy vectors containing a part of an ARS and part of a centromere sequence (CEN); and yeast episomal plasmids (YEp), which are high copy number plasmids comprising a fragment from a 2 micron circle (a natural yeast plasmid) that allows for 50 or more copies to be stably propagated per cell.

In other embodiments, the expression construct comprises a virus or engineered construct derived from a viral genome. A number of viral based systems have been developed for gene transfer into mammalian cells. These include adenoviruses, retroviruses (γ-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60 (2): 249-271; and Lundstrom (2003) Trends Biotechnol. 21 (3): 117-122; herein incorporated by reference in their entireties). The ability of certain viruses to enter cells via receptor-mediated endocytosis, to integrate into host cell genomes and express viral genes stably and efficiently have made them attractive candidates for the transfer of foreign genes into mammalian cells.

For example, retroviruses provide a convenient platform for gene delivery systems. Selected sequences can be inserted into a vector and packaged in retroviral particles. The recombinant virus can then be isolated and delivered to host cells, or cells of a selected subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr. Pharm. Des. 17 (24): 2516-2527). Lentiviruses are a class of retroviruses that are particularly useful for delivering polynucleotides to mammalian cells because they are able to infect both dividing and nondividing cells (see e.g., Lois et a1 (2002) Science 295:868-872; Durand et al. (2011) Viruses 3 (2): 132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering nucleic acids encoding the engineered retrons is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules of interest include those derived from the pox family of viruses, including vaccinia virus and avian poxvirus. By way of example, vaccinia virus recombinants expressing a nucleic acid molecule of interest (e.g., engineered retron) can be constructed as follows. The DNA encoding the particular nucleic acid sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the nucleic acid molecules of interest. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77:10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the nucleic acids of interest (e.g., engineered retron) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the nucleic acid of interest, driven by a T7 promoter. The polymerase expressed in the cytoplasm from the vaccinia virus recombinant transcribes the transfected DNA into RNA. The method provides for high level, transient, cytoplasmic production of large quantities of RNA. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of nucleic acids using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more templates. Concomitantly, there can be modified retron nucleic acids whose expression is under the control of the T7 promoter. Thus, some of the T7 RNA polymerase generated from translation of the amplification template RNA will lead to transcription of the desired retron ncRNAs and/or retron reverse transcriptases. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

Insect cell expression systems, such as baculovirus systems, can also be used and are known to those of skill in the art and described in, e.g., Baculovirus and Insect Cell Expression Protocols (Methods in Molecular Biology, D. W. Murhammer ed., Humana Press, 2^{nd} edition, 2007) and L. King, The Baculovirus Expression System: A laboratory guide (Springer, 1992). Materials and methods for baculovirus/insect cell expression systems are commercially available in kit form from, inter alia, Thermo Fisher Scientific (Waltham, MA) and Clontech (Mountain View, CA).

Plant expression systems can also be used for transforming plant host cells. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

In order to effect expression of engineered retron constructs, the expression construct can be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsulated in an infectious viral particle.

Several non-viral methods for the transfer of expression constructs into cultured cells also are contemplated. These include the use of calcium phosphate precipitation, DEAE-dextran, electroporation, direct microinjection, DNA-loaded liposomes, lipofectamine-DNA complexes, cell sonication, gene bombardment using high velocity microprojectiles, and receptor-mediated transfection (see, e.g., Graham and Van Der Eb (1973) Virology 52:456-467; Chen and Okayama (1987) Mol. Cell Biol. 7:2745-2752; Rippe et al. (1990) Mol. Cell Biol. 10:689-695; Gopal (1985) Mol. Cell Biol. 5:1188-1190; Tur-Kaspa et al. (1986) Mol. Cell. Biol. 6:716-718; Potter et al. (1984) Proc. Natl. Acad. Sci. USA 81:7161-7165); Harland and Weintraub (1985) J. Cell Biol. 101:1094-1099); Nicolau & Sene (1982) Biochim. Biophys. Acta 721:185-190; Fraley et al. (1979) Proc. Natl. Acad. Sci. USA 76:3348-3352; Fechheimer et al. (1987) Proc Natl. Acad. Sci. USA 84:8463-8467; Yang et al. (1990) Proc. Natl. Acad. Sci. USA 87:9568-9572; Wu and Wu (1987) J. Biol. Chem. 262:4429-4432; Wu and Wu (1988) Biochemistry 27:887-892; herein incorporated by reference). Some of these techniques may be successfully adapted for in vivo or ex vivo use.

Delivery of retron nucleic acids to a cell can generally be accomplished with or without vectors. The retrons, retron nucleic acids, or vectors containing them may be introduced into any type of cell, including any cell from a prokaryotic, eukaryotic, or archaeon organism, including bacteria, archaea, fungi, protists, plants (e.g., monocotyledonous and dicotyledonous plants), and animals (e.g., vertebrates and invertebrates). Examples of animal cells that may be transfected with an engineered retron include, without limitation, cells from vertebrates such as fish, birds, mammals (e.g., human and non-human primates, farm animals, pets, and laboratory animals), reptiles, and amphibians. Examples of plant cells that may be transfected with an engineered retron include, without limitation, cells from crops including cereals such as wheat, oats, and rice, legumes such as soybeans and peas, corn, grasses such as alfalfa, and cotton. The engineered retrons can be introduced into a single cell or a population of cells of interest. Cells from tissues, organs, and biopsies, as well as recombinant cells, genetically modified cells, cells from cell lines cultured in vitro, and artificial cells (e.g., nanoparticles, liposomes, polymersomes, or microcapsules encapsulating nucleic acids) may all be transfected with the engineered retrons. The subject methods are also applicable to cellular fragments, cell components, or organelles (e.g., mitochondria in animal and plant cells, plastids (e.g., chloroplasts) in plant cells and algae). Cells may be cultured or expanded after transfection with the engineered retron constructs.

A variety of methods for introducing nucleic acids into a host cell are available. Commonly used methods include chemically induced transformation, typically using divalent cations (e.g., CaCl₂)), dextran-mediated transfection, polybrene mediated transfection, lipofectamine and LT-1 mediated transfection, electroporation, protoplast fusion, encapsulation of nucleic acids in liposomes, and direct microinjection of the nucleic acids comprising engineered retrons into nuclei. See, e.g., Sambrook et al. (2001) Molecular Cloning, a laboratory manual, 3^{rd} edition, Cold Spring Harbor Laboratories, New York, Davis et al. (1995) Basic Methods in Molecular Biology, 2^{nd} edition, McGraw-Hill, and Chu et al. (1981) Gene 13:197; herein incorporated by reference in their entireties.

Once the expression construct has been delivered into the cell the vector or cassette comprising the retron nucleic acids may be positioned and expressed at different sites. In certain embodiments, the vector or cassette comprising the retron nucleic acids may be stably integrated into the genome of the cell. This integration may be in the cognate location and orientation, or it may be integrated in a random, non-specific location (gene augmentation). In yet further embodiments, the vector or cassette comprising the retron nucleic acids may be stably maintained in the cell as a separate, episomal segment of DNA. Such nucleic acid segments or “episomes” encode sequences sufficient to permit maintenance and replication independent of or in synchronization with the host cell cycle. How the vector or cassette comprising the retron nucleic acids are delivered to a cell and where in the cell the nucleic acid remains is dependent on the type of expression construct employed.

In yet another embodiment, the expression construct may simply consist of naked recombinant DNA or plasmids comprising the retron nucleic acids (e.g., expression cassettes). Transfer of the constructs may be performed by any of the methods mentioned above which physically or chemically permeabilize the cell membrane. This is particularly applicable for transfer in vitro but it may be applied to in vivo use as well. Dubensky et al. (Proc. Natl. Acad. Sci. USA (1984) 81:7529-7533) successfully injected polyomavirus DNA in the form of calcium phosphate precipitates into liver and spleen of adult and newborn mice demonstrating active viral replication and acute infection. Benvenisty & Neshif (Proc. Natl. Acad. Sci. USA (1986) 83:9551-9555) also demonstrated that direct intraperitoneal injection of calcium phosphate-precipitated plasmids results in expression of the transfected genes. It is envisioned that DNA encoding retron nucleic acids of interest may also be transferred in a similar manner in vivo and express retron products.

In other cases, a naked DNA expression construct may be transferred into cells by particle bombardment. This method depends on the ability to accelerate DNA-coated microprojectiles to a high velocity allowing them to pierce cell membranes and enter cells without killing them (Klein et al. (1987) Nature 327:70-73). Several devices for accelerating small particles have been developed. One such device relies on a high voltage discharge to generate an electrical current, which in turn provides the motive force (Yang et al. (1990) Proc. Natl. Acad. Sci. USA 87:9568-9572). The microprojectiles may consist of biologically inert substances, such as tungsten or gold beads.

In a further embodiment, the expression construct may be delivered using liposomes. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh & Bachhawat (1991) Liver Diseases, Targeted Diagnosis and Therapy Using Specific Receptors and Ligands, Wu et al. (Eds.), Marcel Dekker, NY, 87-104). Also contemplated is the use of lipofectamine-DNA complexes.

In certain embodiments, the liposome may be complexed with a hemagglutinin virus (HVJ). This has been shown to facilitate fusion with the cell membrane and promote cell entry of liposome-encapsulated DNA (Kaneda et al. (1989) Science 243:375-378). In other embodiments, the liposome may be complexed or employed in conjunction with nuclear non-histone chromosomal proteins (HMG-I) (Kato et al. (1991) J. Biol. Chem. 266 (6): 3361-3364). In yet further embodiments, the liposome may be complexed or employed in conjunction with both HVJ and HMG-I. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993) Adv. Drug Delivery Rev. 12:159-167).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin (see, e.g., Wu and Wu (1987), supra; Wagner et al. (1990) Proc. Natl. Acad. Sci. USA 87 (9): 3410-3414). A synthetic neoglycoprotein, which recognizes the same receptor as ASOR, has been used as a gene delivery vehicle (Ferkol et al. (1993) FASEB J. 7:1081-1091; Perales et al. (1994) Proc. Natl. Acad. Sci. USA 91 (9): 4086-4090), and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (Methods Enzymol. (1987) 149:157-176) employed lactosyl-ceramide, a galactose-terminal asialoganglioside, incorporated into liposomes and observed an increase in the uptake of the insulin gene by hepatocytes. Thus, it is feasible that a nucleic acid encoding a particular gene also may be specifically delivered into a cell by any number of receptor-ligand systems with or without liposomes. Also, antibodies to surface antigens on cells can similarly be used as targeting moieties.

In a particular example, a recombinant polynucleotide comprising retron nucleic acids may be administered in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP: cholesterol or cholesterol derivative formulation that can effectively be used for gene therapy. Other disclosures also discuss different lipid or liposomal formulations including nanoparticles and methods of administration; these include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

### Genomic Editing

The methods described herein can perform genomic editing by using clustered regularly interspaced short palindromic repeats (CRISPR)/CRISPR-associated (Cas) systems. CRISPR/Cas systems are useful, for example, for RNA-programmable genome editing (see e.g., Marraffini and Sontheimer. Nature Reviews Genetics 11:181-190 (2010); Sorek et al. Nature Reviews Microbiology 2008 6:181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12:177-186; all of which are incorporated by reference herein in their entireties).

A CRISPR guide RNA system can be adapted for use in the methods and compositions described herein. Two RNAs can be used in CRISPR genomic editing systems: a CRISPR RNA (crRNA), which is a 17-20 nucleotide sequence complementary to the target DNA, and a trans-activating crRNA (tracrRNA) that is a binding scaffold for the Cas nuclease. In some cases the two RNAs are fused to make a single guide RNA (sgRNA). The tracrRNA forms a stem loop that is recognized and bound by the cas nuclease. The crRNA typically has shorter sequence than the tracrRNA. The term “guide RNA” as used herein refers to either a single guide RNA (sgRNA) or a crRNA. The CRISPR technique is generally described, for example, by Mali et al. Science 339:823-6 (2013); which is incorporated by reference herein in its entirety.

The guide RNA system used herein is encoded within or adjacent to the ncRNA coding region of the expression cassettes. Hence, upon transcription of the guide RNA, it can target a Cas enzyme to the desired location in the genome, where it can cleave the genomic DNA for generation of a genomic modification. Donor DNA encoded within the retron ncRNA and reverse transcribed within the host cells modifies (e.g., repairs) the genomic target site.

There are several types of CRISPR systems, some of which are summarized in the chart below.

CRISPR System Types Overview

| System | Features | Examples |
|---|---|---|
| Type I | Multiple proteins (5-7 proteins | Staphylococcus epidermidis |
| | typical), crRNA. DNA Cleavage | (Type IA) |
| | is catalyzed by Cas3. | |
| Type II | 3-4 proteins (one protein (Cas9) | Streptococcus pyogenes |
| | has nuclease activity) two RNAs. | CRISPR/Cas9, Francisella |
| | Target DNA cleavage catalyzed by | novicida U112 Cpf1 |
| | Cas9 and RNA components. | |
| Type III | Five or six proteins required for | S. epidermidis (Type IIIA); |
| | cutting, number of required for | P. furiosus; (Type IIIB). |
| | RNAs unknown but expected to | |
| | be one. Type IIIB systems have | |
| | the ability to target RNS. | |

In some cases, the cas nuclease is a Type II CRISPR endonuclease. The term “Class II CRISPR endonuclease” refers to endonucleases that have similar endonuclease activity as Cas9 and participate in a Class II CRISPR system. The Cas9 nuclease can, for example, be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

An example of a Class II CRISPR system is the type II CRISPR locus from Streptococcus pyogenes SF370, which contains a cluster of four genes Cas9, Cas1, Cas2, and Csn1, as well as a tracrRNA and a characteristic array of repetitive sequences (direct repeats) interspaced by short stretches of non-repetitive sequences (spacers, about 30 bp each). In this system, targeted DNA double-strand break (DSB) may be generated in four sequential steps. First, the pre-crRNA array and tracrRNA, may be transcribed from the expression cassette that encodes the ncRNA and the guide RNA. Second, tracrRNA may hybridize to the direct repeats of pre-CRISPR guide RNA (pre-crRNA), which is then processed into mature crRNAs containing individual spacer sequences. Third, the mature crRNA: tracrRNA complex can direct Cas9 to the DNA target consisting of the protospacer and the corresponding PAM sequence via heteroduplex formation between the spacer region of the crRNA and the protospacer DNA. Finally, Cas9 may mediate cleavage of target DNA upstream of PAM to create a double-stranded break within the protospacer.

A “guide RNA” or “gRNA” as provided herein refers to a ribonucleotide sequence capable of binding a cas nuclease, thereby forming ribonucleoprotein complex. The gRNA includes a nucleotide sequence complementary to a target site (e.g., near or at a genomic site to be edited). In some cases, the guide RNA includes one or more RNA molecules. TracrRNAs can be used to facilitate assembly of a ribonucleoprotein complex that includes the gRNA together with the tracrRNA and a cas nuclease. A complementary nucleotide sequence of the guide RNA can mediate binding of the ribonucleoprotein complex to the target site thereby providing the sequence specificity of the ribonucleoprotein complex. Thus, the guide RNA includes a sequence that is complementary to a target nucleic acid sequence such that the guide RNA binds a target nucleic acid sequence.

In some cases, the complement of the guide RNA includes a sequence having a sequence identity of about 50%, 55%, 60%, 65%, 70%, 75%, 80%, 85%, 90%, 95%, 96%, 97%, 98% or 99% to a target nucleic acid (e.g., a target genomic DNA sequence). In some cases, a target nucleic acid sequence is a nucleic acid sequence expressed by a cell. In some cases, the target nucleic acid sequence is an exogenous nucleic acid sequence. In some cases, the target nucleic acid sequence is an endogenous nucleic acid sequence. In some cases, the target nucleic acid sequence forms part of a cellular gene. In some cases, the target nucleic acid sequence is a genomic DNA site or location. Thus, some cases, the guide RNA is complementary to a cellular gene or fragment thereof. In some cases, the guide RNA includes a sequence having sequence identity of about 50%, 55%, 60%, 65%, 70%, 75%, 80%, 85%, 90%, 95%, 96%, 97%, 98% or 99% to the target nucleic acid sequence. In some cases, the guide RNA includes a sequence that is about 50%, 55%, 60%, 65%, 70%, 75%, 80%, 85%, 90%, 95%, 96%, 97%, 98% or 99% complementary to the sequence of a cellular gene. In some cases, the guide RNA binds a cellular gene target sequence. In some cases, the guide RNA or complement thereof, includes a sequence having a sequence identity of at least about 90%, 95%, or 100% to a target nucleic acid.

In some cases, segment bound by a guide RNA within the target nucleic acid is about or at least about 10, 15, 20, 25, or more nucleotides in length.

The guide RNA is a single-stranded ribonucleic acid, although in some cases it may form some double-stranded regions by folding onto itself. In some cases, the guide RNA is about 10, 20, 30, 40, 50, 60, 70, 80, 90, 100 or more nucleic acid residues in length. In some cases, the guide RNA is from about 10 to about 30 nucleic acid residues in length. In some cases, the guide RNA is about 20 nucleic acid residues in length. For example, the length of the guide RNA can be at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides or residues in length. In some cases, the guide RNA is from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, or more nucleotides or residues in length. In some cases, the guide RNA is from 10 to 15, 10 to 20, 10 to 30, 10 to 40, or 10 to 50 residues in length.

### Definitions

The term “about” as used herein when referring to a measurable value such as an amount, a length, and the like, is meant to encompass variations of +20% or +10%, more preferably +5%, even more preferably +1%, and still more preferably +0.1% from the specified value.

“Recombinant” as used herein to describe a nucleic acid molecule means a polynucleotide of retron, genomic, cDNA, bacterial, semisynthetic, or synthetic origin which, by virtue of its origin or manipulation, is not associated with all or a portion of the polynucleotide with which it is associated in nature.

The term “recombinant” as used with respect to a protein or polypeptide means a polypeptide produced by expression of a recombinant polynucleotide. In general, the polynucleotide of interest is cloned and then expressed in transformed organisms, for example, as described herein. The host organism expresses the foreign nucleic acids to produce the RNA, RT-DNA, or protein under expression conditions.

As used herein, a “cell” refers to any type of cell isolated from a prokaryotic, eukaryotic, or archaeon organism, including bacteria, archaea, fungi, protists, plants, and animals, including cells from tissues, organs, and biopsies, as well as recombinant cells, cells from cell lines cultured in vitro, and cellular fragments, cell components, or organelles comprising nucleic acids. The term also encompasses artificial cells, such as nanoparticles, liposomes, polymersomes, or microcapsules encapsulating nucleic acids. The methods described herein can be performed, for example, on a sample comprising a single cell or a population of cells. The term also includes genetically modified cells.

The term “transformation” refers to the insertion of an exogenous polynucleotide (e.g., an engineered retron) into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells,” “host cells”, “cells”, “cell lines”, “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide or a selected RNA, is a nucleic acid molecule which is transcribed (in the case of DNA templates) into RNA and/or translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, ncRNAs, tracrRNAs, ncRNAs modified to include heterologous sequences, cDNA from viral, prokaryotic or eukaryotic ncRNA, mRNA, genomic DNA sequences from retron, viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper polymerases are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide or RNA sequence. For example, the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. The RNA sequence or a portion thereof contains a nucleotide sequence of at least 3 to 5 nucleotides, more preferably at least 8 to 10 nucleotides, and even more preferably at least 15 to 20 nucleotides.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein, DNA, or RNA or cause other adverse consequences. That is, a nucleic acid or peptide of this invention is purified if it is substantially free of cellular material, viral material, or culture medium when obtained from nature or when produced by recombinant DNA techniques, or free from chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

“Substantially purified” generally refers to isolation of a substance (nucleic acid, compound, polynucleotide, protein, polypeptide, peptide composition) such that the substance comprises the majority percent of the sample in which it resides. Typically, in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

“Purified polynucleotide” refers to a polynucleotide of interest or fragment thereof which is essentially free, e.g., contains less than about 50%, preferably less than about 70%, and more preferably less than about at least 90%, of the protein and/or nucleic acids with which the polynucleotide is naturally associated. Techniques for purifying polynucleotides of interest are available in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally available. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material and includes uptake of peptide-linked or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Expression” refers to detectable production of a gene product by a cell. The gene product may be a transcription product (i.e., RNA), which may be referred to as “gene expression”, or the gene product may be a translation product of the transcription product (i.e., a protein), depending on the context.

“Mammalian cell” refers to any cell derived from a mammalian subject suitable for transfection with retron nucleic acids or vector systems comprising retron nucleic acids, as described herein. The cell may be xenogeneic, autologous, or allogeneic. The cell can be a primary cell obtained directly from a mammalian subject. The cell may also be a cell derived from the culture and expansion of a cell obtained from a mammalian subject. Immortalized cells are also included within this definition. In some embodiments, the cell has been genetically engineered to express a recombinant protein and/or nucleic acid.

The term “subject” includes animals, including both vertebrates and invertebrates, including, without limitation, invertebrates such as arthropods, mollusks, annelids, and cnidarians; and vertebrates such as amphibians, including frogs, salamanders, and caecillians; reptiles, including lizards, snakes, turtles, crocodiles, and alligators; fish; mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, geese, and the like. In some cases, the disclosed methods find use in experimental animals, in veterinary application, and in the development of animal models for disease, including, but not limited to, rodents including mice, rats, and hamsters; primates, and transgenic animals.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide or nucleic acid “derived from” a designated sequence refers to a polynucleotide or nucleic acid that includes a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

A “barcode” refers to one or more nucleotide sequences that are used to identify a nucleic acid or cell with which the barcode is associated. Barcodes can be 3-1000 or more nucleotides in length, preferably 10-250 nucleotides in length, and more preferably 10-50 nucleotides in length, including any length within these ranges, such as 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, or 1000 nucleotides in length. Barcodes may be used, for example, to identify a single cell, subpopulation of cells, colony, or sample from which a nucleic acid originated. Barcodes may also be used to identify the identity, presence or position (i.e., positional barcode) of a nucleic acid, cell, colony, or sample from which a nucleic acid originated, such as the position of an insertion into a genome, a colony in a cellular array, the presence of donor DNA in a cell. For example, a barcode may be used to identify a genetically modified cell having a donor DNA encoded by a modified ncRNA. In some embodiments, a barcode is used to identify a particular type of genome edit or a particular type of donor nucleic acid.

The terms “hybridize” and “hybridization” refer to the formation of complexes between nucleotide sequences which are sufficiently complementary to form complexes via Watson-Crick base pairing.

The term “homologous region” refers to a region of a nucleic acid with homology to another nucleic acid region. Thus, whether a “homologous region” is present in a nucleic acid molecule is determined with reference to another nucleic acid region in the same or a different molecule. Further, since a nucleic acid is often double-stranded, the term “homologous, region,” as used herein, refers to the ability of nucleic acid molecules to hybridize to each other. For example, a single-stranded nucleic acid molecule can have two homologous regions which are capable of hybridizing to each other. Thus, the term “homologous region” includes nucleic acid segments with complementary sequences. Homologous regions may vary in length but will typically be between 4 and 500 nucleotides (e.g., from about 4 to about 40, from about 40 to about 80, from about 80 to about 120, from about 120 to about 160, from about 160 to about 200, from about 200 to about 240, from about 240 to about 280, from about 280 to about 320, from about 320 to about 360, from about 360 to about 400, from about 400 to about 440, etc.).

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands can base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. However, when uracil is denoted in the context of the present invention, the ability to substitute a thymine is implied, unless otherwise stated. “Complementarity” may exist between two RNA strands, two DNA strands, or between an RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art.

The term “Cas9” as used herein encompasses type II clustered regularly interspaced short palindromic repeats (CRISPR) system Cas9 endonucleases from any species, and also includes biologically active fragments, variants, analogs, and derivatives thereof that retain Cas9 endonuclease activity (i.e., catalyze site-directed cleavage of DNA to generate double-strand breaks). A Cas9 endonuclease binds to and cleaves DNA at a site comprising a sequence complementary to its bound guide RNA (gRNA). For purposes of Cas9 targeting, a gRNA may comprise a sequence “complementary” to a target sequence (e.g., major or minor allele), capable of sufficient base-pairing to form a duplex (i.e., the gRNA hybridizes with the target sequence). Additionally, the gRNA may comprise a sequence complementary to a PAM sequence, wherein the gRNA also hybridizes with the PAM sequence in a target DNA.

The term “donor polynucleotide” or “donor DNA” refers to a nucleic acid or polynucleotide that provides a nucleotide sequence of an intended edit to be integrated into the genome at a target locus by HDR or recombineering.

A “target site” or “target sequence” is the nucleic acid sequence recognized (i.e., sufficiently complementary for hybridization) by a guide RNA (gRNA) or a homology arm of a donor polynucleotide (donor DNA). The target site may be allele-specific (e.g., a major or minor allele). For example, a target site can be a genomic site that is intended to be modified such as by insertion of one or more nucleotides, replacement of one or more nucleotides, deletion of one or more nucleotides, or a combination thereof.

By “homology arm” is meant a portion of a donor polynucleotide that is responsible for targeting the donor polynucleotide to the genomic sequence to be edited in a cell. The donor polynucleotide typically comprises a 5′ homology arm that hybridizes to a 5′ genomic target sequence and a 3′ homology arm that hybridizes to a 3′ genomic target sequence flanking a nucleotide sequence comprising the intended edit to the genomic DNA. The homology arms are referred to herein as 5′ and 3′ (i.e., upstream and downstream) homology arms, which relates to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively. For example, the nucleotide sequence comprising the intended edit can be integrated into the genomic DNA by HDR or recombineering at the genomic target locus recognized (i.e., sufficiently complementary for hybridization) by the 5′ and 3′ homology arms.

In general, “a CRISPR system” refers collectively to transcripts and other elements involved in the expression of or directing the activity of CRISPR-associated (“Cas”) genes, including sequences encoding a Cas gene, and a CRISPR array nucleic acid sequence including a leader sequence and at least one repeat sequence. In some embodiments, one or more elements of a CRISPR system are derived from a type I, type II, or type III CRISPR system. Cas1 and Cas2 are found in all three types of CRISPR-Cas systems, and they are involved in spacer acquisition. In the I-E system of E. coli, Cas1 and Cas2 form a complex where a Cas2 dimer bridges two Cas1 dimers. In this complex Cas2 performs a non-enzymatic scaffolding role, binding double-stranded fragments of invading DNA, while Cas1 binds the single-stranded flanks of the DNA and catalyzes their integration into CRISPR arrays.

In some embodiments, one or more elements of a CRISPR system are derived from a particular organism comprising an endogenous CRISPR system, such as Streptococcus pyogenes. In general, a CRISPR system can be characterized by elements that promote the formation of a CRISPR complex at the site of a target sequence (also referred to as a protospacer in the context of an endogenous CRISPR system).

In some embodiments, a vector comprises a regulatory element operably linked to an enzyme-coding sequence encoding a CRISPR enzyme, such as a Cas protein. Non-limiting examples of Cas proteins include Cas1, CaslB, Cas2, Cas3, Cas4, Cas5, Cas6, Cas7, Cas8, Cas9 (also known as Csn1 and Csx12), CasIO, Csy1, Csy2, Csy3, Cse1, Cse2, Cscl, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, CsxlO, Csx16, CsaX, Csx3, Csx1, Csx15, Csf1, Csf2, Csf3, Csf4, homologs thereof, or modified versions thereof.

In certain embodiments, the disclosure provides protospacers that are adjacent to short (3-5 bp) DNA sequences termed protospacer adjacent motifs (PAM). The PAMs are important for type I and type II systems during acquisition. In type I and type II systems, protospacers are excised at positions adjacent to a PAM sequence, with the other end of the spacer is cut using a ruler mechanism, thus maintaining the regularity of the spacer size in the CRISPR array. The conservation of the PAM sequence differs between CRISPR-Cas systems and may be evolutionarily linked to Cas1 and the leader sequence.

In one embodiment, the protospacer is a defined synthetic DNA. In some embodiments, the defined synthetic DNA is at least 3, 5, 10, 20, 30, 40, or 50 nucleotides, or between 3-50, or between 10-100, or between 20-90, or between 30-80, or between 40-70, or between 50-60, nucleotides in length. In one embodiment, the oligo nucleotide sequence or the defined synthetic DNA includes a modified “AAG” protospacer adjacent motif (PAM).

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et a1, J. BacterioL, 169:5429-5433 (1987); and Nakata et al., J. BacterioL, 171:3553-3556 (1989)), and associated genes. Similar interspersed SSRs have been identified in Haloferax mediterranei, Streptococcus pyogenes, Anabaena, and Mycobacterium tuberculosis (See, Groenen et al., Mol. Microbiol., 10:1057-1065 (1993); Hoe et al., Emerg. Infect. Dis., 5:254-263 (1999); Masepohl et a1, Biochim. Biophys. Acta 1307:26-30 (1996); and Mojica et a1, Mol. Microbiol, 17:85-93 (1995)). The CRISPR loci typically differ from other SSRs by the structure of the repeats, which have been termed short regularly spaced repeats (SRSRs) (Janssen et a1, OMICS J. Integ. Biol., 6:23-33 (2002); and Mojica et a1, Mol. Microbiol., 36:244-246 (2000)). In general, the repeats are short elements that occur in clusters that are regularly spaced by unique intervening sequences with a substantially constant length (Mojica et al., (2000), supra). Although the repeat sequences are highly conserved between strains, the number of interspersed repeats and the sequences of the spacer regions typically differ from strain to strain (van Embden et al., J. Bacteriol., 182:2393-2401 (2000)). CRISPR loci have been identified in more than 40 prokaryotes (See e.g., Jansen et a1, Mol. Microbiol., 43:1565-1575 (2002); and Mojica et a1, (2005)) including, but not limited to Aeropyrum, Pyrobaculum, Sulfolobus, Halocarcula, Archaeoglobus, Methanobacteriumn, Methanococcus, Methanosarcina, Methanopyrus, Pyrococcus, Picrophilus, Thernioplasnia, Corynebacterium, Mycobacterium, Streptomyces, Aquifrx, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In some embodiments, an enzyme coding sequence encoding a CRISPR enzyme (e.g., cas9) is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about one or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

“Administering” a nucleic acid, such as an expression cassette, engineered retron construct or vector comprising an expression cassette or engineered retron construct to a cell comprises transducing, transfecting, electroporating, translocating, fusing, phagocytosing, shooting or ballistic methods, etc., i.e., any means by which a nucleic acid can be transported across a cell membrane.

The subject matter disclosed herein is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosed subject matter. The upper and lower limits of these smaller ranges may independently be included in the smaller ranges, and are also encompassed within the disclosed subject matter, subject to any specifically excluded limit in the stated range. Where the stated range includes one or both of the limits, ranges excluding either or both of those included limits are also included in the disclosed subject matter.

Unless defined otherwise, all technical and scientific terms used herein have the same meaning as commonly understood by one of ordinary skill in the art to which the disclosed subject matter belongs. Although any methods and materials similar or equivalent to those described herein can also be used in the practice or testing of the disclosed subject matter, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of any features or elements described herein, which includes use of a “negative” limitation.

It is appreciated that certain features of the disclosed subject matter, which are, for clarity, described in the context of separate embodiments, may also be provided in combination in a single embodiment. Conversely, various features of the disclosed subject matter, which are, for brevity, described in the context of a single embodiment, may also be provided separately or in any suitable sub-combination. All combinations of the embodiments pertaining to the disclosure are specifically embraced by the disclosed subject matter and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the disclosed subject matter is not entitled to antedate such publication. Further, the dates of publication provided may be different from the actual publication dates which may need to be independently confirmed.

The following Examples illustrate some of the materials, methods, and experiments that were used or performed in the development of the invention.

### Example 1: Methods for Analyzing Retron Systems for Genomic Editing

This example illustrates analysis of a library of retron variants, using bar code tags to locate, sequence, and measure the relative integration frequency of the different variants.

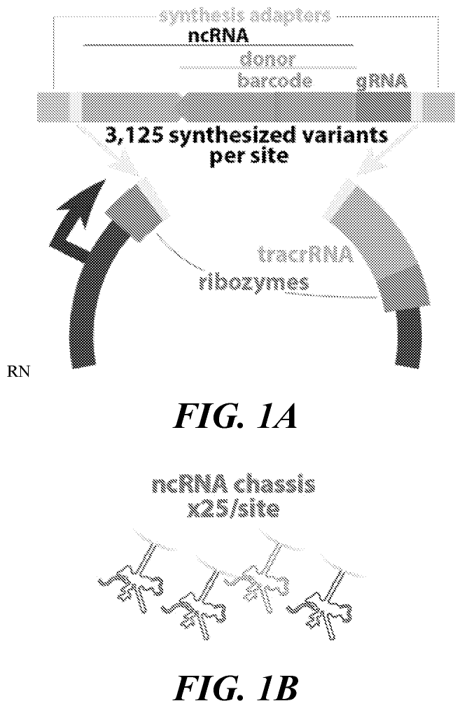

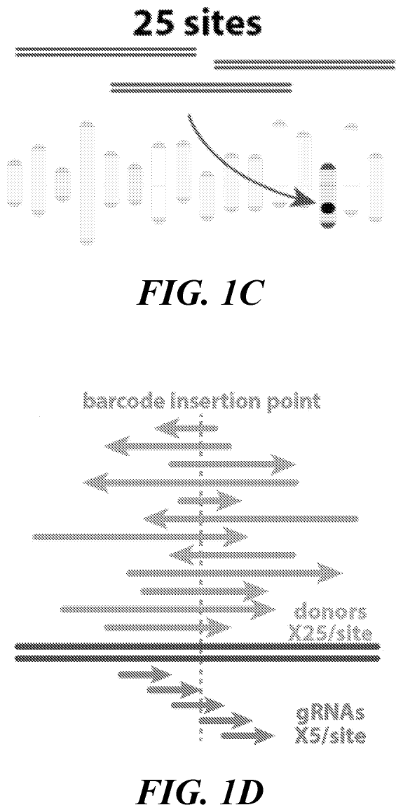

FIG. 1A is a schematic diagram of a retron variant construct that encodes a variant retron ncRNA, linked to a gRNA coding region with adaptors flanking the coding regions. The adaptors are useful inter alia for library construction. The variant retron nucleic acids can be inserted into an expression vector using restriction sites within the adaptors. Such insertion can retain or eliminate the adaptor sequences. The construct can include tracrRNA coding regions so that tracrRNAs are transcribed when the retron ncRNA is transcribed.

A library was designed to include 3125 variant constructs. Such construct were designed to evaluate the effect of different ncRNA/donor DNA/gRNA combinations, and different genomic site target site sequences in parallel. The variants included about 25 different target sites in yeast and human genomes. For each inserted site, precise retron-based editors were designed with 25 different donor sequences spanning the site, 5 different gRNAs spanning the site, and 25 different optimized ncRNA chasses. The ncRNA chasses were selected in preliminary experiments to exhibit high productions of RT-DNA that served as donor DNAs for genomic editing.

All combinations of these elements were synthesized for each site (3,125 variations per site for 78,125 total variations), and in some experiments, the elements were each designed to include a unique barcode. For example, a donor DNA can include a bar code that can be inserted into the genome at a specific location within the insertion site of the DNA to be edited. Such donor DNA/barcode insertion facilitates analysis of the editing performance of different donor DNA/gRNAs at the genomic site to be edited (FIG. 1D). After genomic editing was allowed to proceed, barcoded sites were sequenced to determine the relative performance of each variant. Improved variant constructs had their barcodes overrepresented in the sequencing results (FIG. 1B-1D).

### Example 2: ncRNA/RT-DNA Features that can be Modified

The methods described herein can be used to evaluate features of the ncRNA that can be modified.

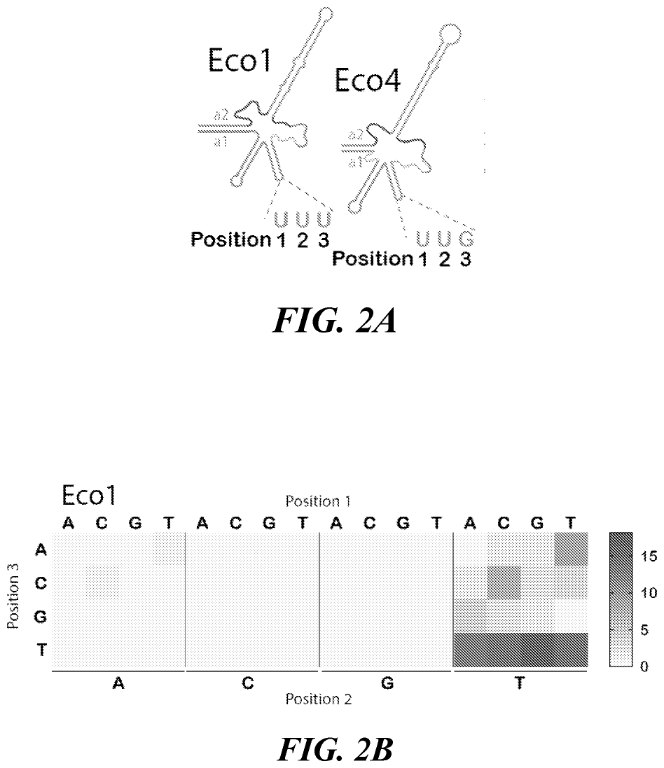

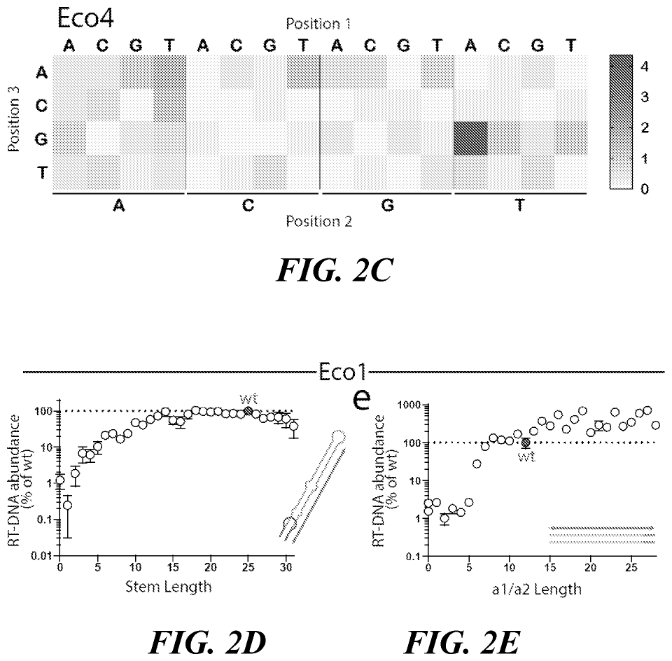

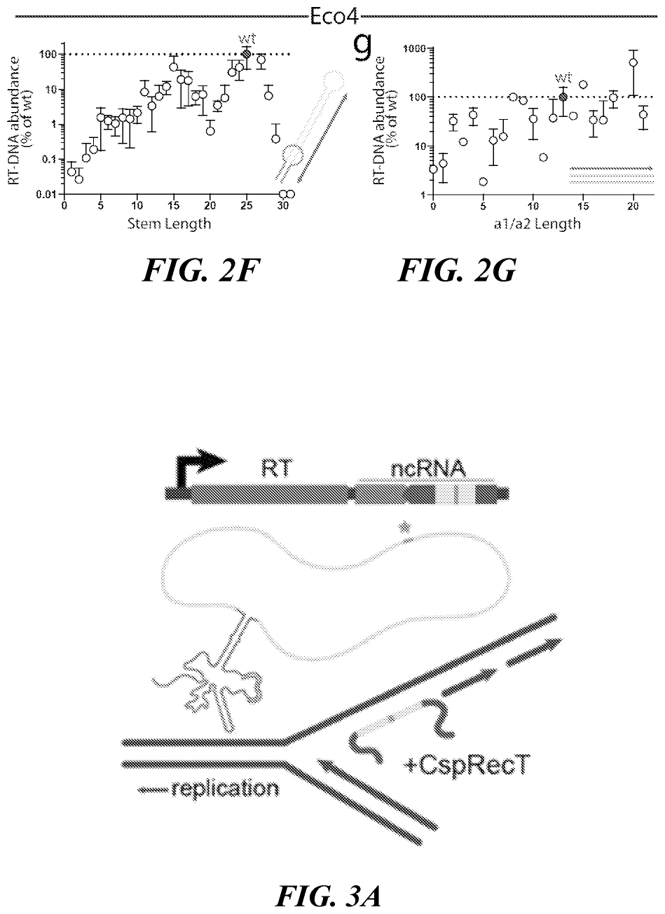

In one experiment, a loop region of the ncRNA was analyzed that was hypothesized to be involved in reverse transcriptase recognition. This loop region had a sequence that was somewhat different in Eco1 and Eco4 retrons (FIG. 2A). As illustrated in FIGS. 2A-2C, this loop was determined to be much more sensitive to modifications in Eco1 than Eco4.

In another experiment, the length of stem regions was evaluated to ascertain optimal stem lengths for retron ncRNAs. As shown in FIGS. 2D-2G, RT-DNA production from both Eco1 and Eco4 ncRNAs was negatively affected by reducing the stem length below about 15 base pairs and by reducing the length of the complementary region at the 5′ and 3′ ends of the ncRNA, termed a1/a2, below about 10 base pairs.

Interestingly, further experiments have shown that extension of the a1/a2 region can result in more than a ten-fold increase in RT-DNA production, which is the improvement that can be used to increase editing rates, for example, in a variety of cell types, including yeast.

The analytic procedures described herein can also be used to identify and quantify modifications of the protein components of the system, such as the retron reverse transcriptase. To do so, a barcode in the msd region can be linked to each modification of the reverse transcriptase gene. Many variant plasmids can be then run in parallel and sequencing or determining the relative abundance of the barcoded RT-DNA can be used to determine the effect of the mutations on RT-DNA production by the variant reverse transcriptases. These experiments have been completed in E. coli but can also be performed in mammalian cells and yeast.

### Example 3: Quantification of Editing Rates Using Modified Retron Editing Templates

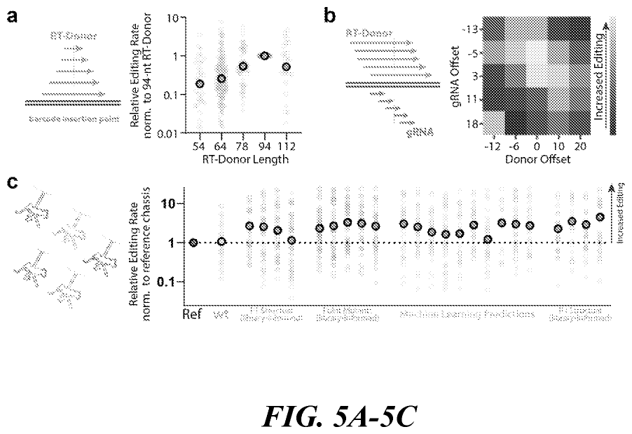

FIG. 5A provides relative editing of variants based on RT-Donor length. Small circles are individual variants, each normalized to a 94 length RT-Donor in sets of variants matched on all other parameters. Large circles are geometric mean of the variants. FIG. 5B provides relative editing of variants based on the RT-Donor and gRNA offset around the barcode insertion point (orange dotted line). FIG. 5C provides relative editing of variants based on ncRNA chassis modifications. Small circles are individual variants, each normalized to a reference chassis (Ref) that was the current standard Editron chassis prior to these experiments, in sets of variants matched on all other parameters. Large circles are geometric mean of the variants.

```
site =
‘AGCTAGACTTGTTTACTTTGTATTTATTAGTATTTGCCTCACATGAGAACACACA
CTCTCCATCCTGCGTACGCCATCCCCTTCGATGCGAGTGCGAATCGCGGCGTCTT
GGTAGCACGGCAGACTGGCTCACTGGACGAAGAGTGCATCCGAGAACCAGAAAC
AAGCAACGTTCCAGTGAGTTGTTCCACACATCCTTTAAAGTTTAGCGTATAGAAA
ATTCGAGAC’ (SEQ ID NO: 10)
#site gRNAs as dictionary where the key is the position of the PAM GG
site_gRNAs= {102:[‘CGATGCGAGTGCGAATCG,‘PAM_-13’](SEQ ID NO: 11),
                                  110:[‘GTGCGAATCGCGGCGTCT’,‘PAM_-5’](SEQ ID NO: 12),
                                  118:[‘CGCGGCGTCTTGGTAGCA’,‘PAM_-3’](SEQ ID NO: 13),
                                  126:[‘CTTGGTAGCACGGCAGAC’,‘PAM_11’](SEQ ID NO: 14),
                                  134:[‘CACGGCAGACTGGCTCAC’,‘PAM_19’]}(SEQ ID NO: 15)
#different chassis, split into two around the donor, except for 22-25,
which are split into three (predonor, extra left stem, and postdonor)
chassis=
{1:[‘TGCGCACCCTTAGCGAGAGGTTTATCATTAAGGTCAACCTCTGGATGTTGTTT
CGGCATCCTGCATTGAATCTGAGTTACTGTCTGTTTTCCT’,‘AGGAAACCCGTTTTT
TCTGACGTAAGGGTGCGCA’],
#1_ec86wt msr/msd (RNA) bare_true_wt (SEQ ID NO: 16)
2:[‘TGCGCACCCTTAGCGAGAGGTTTATCATTAAGGTCAACCTCTGGATGTTGTTT
CGGCATCCTGCATTGAATCTGAGTTACTGTCTGTTTTCCT’,‘AGGAAACCCGTTTCT
TCTGACGTAAGGGTGCGCA’],
#2_ec86wt msr/msd (RNA) bare_CRISPEY_wt (SEQ ID NO: 17)
3:[‘TGATAAGATTCCGTATGCGCACCCTTAGCGAGAGGTTTATCATTAAGGTCAAC
CTCTGGATGTTGTTTCGGCATCCTGCATTGAATCTGAGTTACTGTCTGTTTTCCT’,
AGGAAACCCGTTTCTTCTGACGTAAGGGTGCGCATACGGAATCTTATCA’],
#3_ec86wt msr/msd (RNA) bare_CRISPEY_Pext27 (SEQ ID NO: 18)
4:[‘AAGATTCCGTATGCGCACCCTTAGCGAGAGGTTTATCATTAAGGTCAACCTCT
GGATGTTGTTTCGGCATCCTGCATTGAATCTGAGTTACTGTCTGTTTTCCT’,
‘AGGAAACCCGTTTCTTCTGACGTAAGGGTGCGCATACGGAATCTT’],
#4_ec86wt msr/msd (RNA) bare_CRISPEY_Pext23 (SEQ ID NO: 19)
5:[‘TTCCGTATGCGCACCCTTAGCGAGAGGTTTATCATTAAGGTCAACCTCTGGAT
GTTGTTTCGGCATCCTGCATTGAATCTGAGTTACTGTCTGTTTTCCT’,‘AGGAAACC
CGTTTCTTCTGACGTAAGGGTGCGCATACGGAA’],
#5_ec86wt msr/msd (RNA) bare CRISPEY_Pext19 (SEQ ID NO: 20)
6:[‘CGTATGCGCACCCTTAGCGAGAGGTTTATCATTAAGGTCAACCTCTGGATGTT
GTTTCGGCATCCTGCATTGAATCTGAGTTACTGTCTGTTTTCCT’,‘AGGAAACCCGT
TTCTTCTGACGTAAGGGTGCGCATACG’],
#6_ec86wt msr/msd (RNA) bare CRISPEY_Pext16 (SEQ ID NO: 21)
7:[‘TGATAAGATTCCGTATGCGCACCCTTAGCGAGAGGTTTATCATTAAGGTCAAC
CTCTGGATGTTGTTTCGGCATCCTGCATTGAATCTGAGTTACTGTCTGTTTTCCT’,
‘GGAAACCCGTTTCTTCTGACGTAAGGGTGCGCATACGGAATCTTATCA’],
#7_ec86wt msr/msd (RNA) bare_CRISPEY_Pext27_d136 (SEQ ID NO: 22)
8:[‘TGATAAGATTCCGTATGCGCACCCTTAGCGAGAGGTTTATCATTAAGGTCAAC
CTCTGGATGTTGTTTCGGCATCCTGCATTGAATCTGAGTTACTGTCTGTTTTCCT’,
‘AGGACCCGTTTCTTCTGACGTAAGGGTGCGCATACGGAATCTTATCA’], #8_ec86wt
msr/msd (RNA) bare_CRISPEY_Pext27_d139 (SEQ ID NO: 23)
9:[‘TGATAAGATTCCGTATGCGCACCCTTAGCGAGAGGTTTATCATTAAGGTCAAC
CTCTGGATGTTGTTTCGGCATCCTGCATTGAATCTGAGTTACTGTCTGTTTTCCT’,
‘AGGAACCGTTTCTTCTGACGTAAGGGTGCGCATACGGAATCTTATCA’], #9_ec86wt
msr/msd (RNA) bare_CRISPEY_Pext27_d141 (SEQ ID NO: 24)
10:[‘TGATAAGATTCCGTATGCGCACCCTTAGCGAGAGGTTTATCATTAAGGTCAA
CCTCTGGATGTTGTTTCGGCATCCTGCATTGAATCTGAGTTACTGTCTGTTTTCCT’,
‘AGGAAACCTGTTTCTTCTGACGTAAGGGTGCGCATACGGAATCTTATCA’],
#10_ec86wt msr/msd (RNA) bare_CRISPEY_Pext27_144T_old11 (SEQ ID NO: 25)
11:[‘TGATAAGATTCCGTATGCGCACCCTTAGCGAGAGGTTTATCATTAAGGTCAA
CCTCTGGATGTTGTTTCGGCATCCTGCATTGAATCTGAGTTACTGTCTGTTTTCCT’,
‘AGGAAACCCGTATCTTCTGACGTAAGGGTGCGCATACGGAATCTTATCA’],
#11_ec86wt msr/msd (RNA) bare_CRISPEY_Pext27_147A_old12 (SEQ ID NO: 26)
12:[‘TGATAAGATTCCGTATGCGCACCCTTAGCGAGAGGTTTATCATTAAGGTCAA
CCTCTGGATGTTGTTTCGGCATCCTGCATTGAATCTGAGTTACTGTCTGTTTTCCT’,
‘TATCATGTAATACTCTACGGCGTAAGGGTGCGCATACGGAATCTTATCA’],
#12_compN0_328 (SEQ ID NO: 27)
13:[‘TGATAAGATTCCGTATGCGCACCCTTAGCGAGAGGTTTATCATTAAGGTCAA
CCTCTGGATGTTGTTTCGGCATCCTGCATTGAATCTGAGTTACTGTCTGTTTTCCT’,
‘ATATCGGCGTAATTTCGGTTCGTAAGGGTGCGCATACGGAATCTTATCA’],
#13_compN0_64294 (SEQ ID NO: 28)
14:[‘TGATAAGATTCCGTATGCGCACCCTTAGCGAGAGGTTTATCATTAAGGTCAA
CCTCTGGATGTTGTTTCGGCATCCTGCATTGAATCTGAGTTACTGTCTGTTTTCCT’,
‘ATCTATCGTGATATTCAGATCGTAAGGGTGCGCATACGGAATCTTATCA’],
#14_compN0_185887 (SEQ ID NO: 29)
15:[‘TGATAAGATTCCGTATGCGCACCCTTAGCGAGAGGTTTATCATTAAGGTCAA
CCTCTGGATGTTGTTTCGGCATCCTGCATTGAATCTGAGTTACTGTCTGTTTTCCT’,
‘ATATGGACACGGTGAGCACTCGTAAGGGTGCGCATACGGAATCTTATCA’],
#15_compN0_232445 (SEQ ID NO: 30)
16:[‘TGATAAGATTCCGTATGCGCACCCTTAGCGAGAGGTTTATCATTAAGGTCAA
CCTCTGGATGTTGTTTCGGCATCCTGCATTGAATCTGAGTTACTGTCTGTTTTCCT’,
‘ATCATTTACGGTACGATCAGCGTAAGGGTGCGCATACGGAATCTTATCA’],
#16_compN0_101299 (SEQ ID NO: 31)
17:[‘TGATAAGATTCCGTATGCGCACCCTTAGCGAGAGGTTTATCATTAAGGTCAA
CCTCTGGATGTTGTTTCGGCATCCTGCATTGAATCTGAGTTACTGTCTGTTTTCCT’,
‘AGGAAATCCAACATTCACTGCGTAAGGGTGCGCATACGGAATCTTATCA’],
#17_compN1_446844 (SEQ ID NO: 32)
18:[‘TGATAAGATTCCGTATGCGCACCCTTAGCGAGAGGTTTATCATTAAGGTCAA
CCTCTGGATGTTGTTTCGGCATCCTGCATTGAATCTGAGTTACTGTCTGTTTTCCT’,
‘GGATCACTCACTGTATACCGCGTAAGGGTGCGCATACGGAATCTTATCA’],
#18_compN1_40329 (SEQ ID NO: 33)
19:[‘TGATAAGATTCCGTATGCGCACCCTTAGCGAGAGGTTTATCATTAAGGTCAA
CCTCTGGATGTTGTTTCGGCATCCTGCATTGAATCTGAGTTACTGTCTGTTTTCCT’,
‘AGGAAACTGTAAATAAACCGCGTAAGGGTGCGCATACGGAATCTTATCA’],
#19_compN1_461043 (SEQ ID NO: 34)
20:[‘TGATAAGATTCCGTATGCGCACCCTTAGCGAGAGGTTTATCATTAAGGTCAA
CCTCTGGATGTTGTTTCGGCATCCTGCATTGAATCTGAGTTACTGTCTGTTTTCCT’,
‘GCGGGCGGTGTAACGTTCCACGTAAGGGTGCGCATACGGAATCTTATCA’],
#20_compN1_25575 (SEQ ID NO: 35)
21:[‘TGATAAGATTCCGTATGCGCACCCTTAGCGAGAGGTTTATCATTAAGGTCAA
CCTCTGGATGTTGTTTCGGCATCCTGCATTGAATCTGAGTTACTGTCTGTTTTCCT’,
‘AGGAAACTGTAGATAACCCGCGTAAGGGTGCGCATACGGAATCTTATCA’],
#21_compN1_470993 (SEQ ID NO: 36)
22:[‘TGATAAGATTCCGTATGCGCACCCTTAGCGAGAGGTTTATCATTAAGGTCAA
CCTCTGGATGTTGTTTCGGCATCCTGCATTGAATCTGAGTTACTGTCTGTTTTCCT’,
‘TG’,‘CCAGGAAACCCGTTTCTTCTGACGTAAGGGTGCGCATACGGAATCTTATCA’],
#22_ec86wt msr/msd (RNA) bare_CRISPEY_Pext27_stem8_old13 (SEQ ID NO: 37)
23: [‘TGATAAGATTCCGTATGCGCACCCTTAGCGAGAGGTTTATCATTAAGGTCAA
CCTCTGGATGTTGTTTCGGCATCCTGCATTGAATCTGAGTTACTGTCTGTTTTCCT’,
‘TGTT’,‘AACCAGGAAACCCGTTTCTTCTGACGTAAGGGTGCGCATACGGAATCTTATCA’],
#23_ec86wt msr/msd (RNA) bare_CRISPEY_Pext27 stem 10_old14 (SEQ ID NO: 38)
24:[‘TGATAAGATTCCGTATGCGCACCCTTAGCGAGAGGTTTATCATTAAGGTCAA
CCTCTGGATGTTGTTTCGGCATCCTGCATTGAATCTGAGTTACTGTCTGTTTTCCT’,
‘TGTTGG’,‘CCAACCAGGAAACCCGTTTCTTCTGACGTAAGGGTGCGCATACGGAATCTTATCA’],
#24_ec86wt msr/msd (RNA) bare_CRISPEY_Pext27_stem 12_old15 (SEQ ID NO: 39)
25:[‘TGATAAGATTCCGTATGCGCACCCTTAGCGAGAGGTTTATCATTAAGGTCAA
CCTCTGGATGTTGTTTCGGCATCCTGCATTGAATCTGAGTTACTGTCTGTTTTCCT’,
‘TGTTGGAA’,‘AGCCAACCAGGAAACCCGTTTCTTCTGACGTAAGGGTGCGCATACGGAATCTTATCA’]}
#25_ec86wt msr/msd (RNA) bare_CRISPEY_Pext27 stem14 old16 (SEQ ID NO: 40)
```

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method comprising: (a) transforming a population of host cells, each host cell comprising a reverse transcriptase and a Cas nuclease, with a library of expression cassettes, each expression cassette comprising a promoter operably linked to a nucleic acid segment encoding a modified retron ncRNA comprising a sequence for a barcode, a sequence for a donor DNA, and sequence for a guide RNA, and; (b) sequencing genomic sites comprising the barcodes within the host cells to determine (i) the identity and frequency of the barcodes in the population, (ii) the sequences of genomic edits made in the host cells, or (iii) a combination thereof.

2. The method of claim 1, which does not comprise quantifying reverse transcribed DNA (RT-DNA) in the host cells.

3. The method of claim 1, wherein the barcodes are within the donor DNAs of one or more of the ncRNAs encoded by expression cassettes.

4. The method of claim 1, wherein each barcode is a marker identifying a particular expression cassette.

5. (canceled)

6. The method of claim 1, wherein one or more of the sequences for the donor DNAs comprise one or more variant nucleotides compared to a genomic DNA target site sequence.

7. (canceled)

8. The method of claim 1, wherein one or more donor DNAs, each comprising a barcode, are incorporated into genomic target sites within one or more host cells.

9. (canceled)

10. The method of claim 1, wherein a subpopulation of host cells is transformed with a series of expression cassettes, each expression cassette comprising a donor DNA designed to edit the same genomic target site, wherein one or more of the series of expression cassettes encodes different guide RNA(s).

11. The method of claim 1, wherein a subpopulation of host cells is transformed with a series of expression cassettes, each expression cassette comprising a different donor DNA designed to edit the same genomic target site.

12. The method of claim 1, wherein one or more sub-populations of the host cells comprise a different Cas nuclease than the other host cells.

13. The method of claim 1, wherein one or more sub-populations of the host cells comprise a different reverse transcriptase than the other host cells.

14. (canceled)

15. The method of claim 1, wherein one or more expression cassettes further comprises at least one or two adapters, restriction sites, or a combination thereof.

16. The method of claim 1, wherein each expression cassette further encodes a trans-activating crRNA (tracrRNA) that is expressed in the host cell(s).

17. (canceled)

18. (canceled)

19. (canceled)

20. (canceled)

21. (canceled)

22. (canceled)

23. (canceled)

24. (canceled)

25. (canceled)

26. The method of claim 1, wherein integration frequencies are determined as the percent of host cells in the population that comprise each type of barcode.

27. An expression cassette comprising a promoter operably linked to a nucleic acid segment encoding a modified retron ncRNA comprising a sequence for a barcode, a sequence for a donor DNA, and sequence for a guide RNA.

28. The of expression cassette claim 27, wherein the barcode is within the sequence for the donor DNA.

29. The expression cassette of claim 27, wherein each barcode is a marker identifying a particular donor DNA.

30. (canceled)

31. The expression cassette of claim 27, wherein the sequence for the donor DNA comprises one or more variant nucleotides compared to a genomic DNA target site sequence.

32. (canceled)

33. The expression cassette of claim 27, wherein the expression cassette further comprises at least one or two adapters, restriction sites, or a combination thereof.

34. The expression cassette of claim 27, wherein the expression cassette further encodes a trans-activating crRNA (tracrRNA).

35. The expression cassette of claim 27, wherein each barcode is a marker identifying a specific ncRNA variant.
